(19) United States
(12) Patent Application Publication
(10) Pub. No.: US 2019/0328770 A1
(43) Pub. Date: Oct. 31, 2019

(54) COMPOSITIONS OF SMAD7 ANTISENSE OLIGONUCLEOTIDE AND METHODS OF TREATING OR PREVENTING PSORIASIS

(71) Applicant: Nogra Pharma Limited, Dublin 2 (IE)

(72) Inventors: Giovanni Monteleone, Grottaferrata (IT); Salvatore Bellinvia, Mendrisio (CH)

(21) Appl. No.: 16/474,894

(22) PCT Filed: Dec. 29, 2017
(86) PCT No.: PCT/EP2017/084814

§ 371 Date: Jun. 28, 2019

Related U.S. Application Data
(60) Provisional application No. 62/440,779, filed on Dec. 30, 2016.

(51) Int. Cl. A61K 31/7125; C12N 15/113

(52) U.S. Cl. CPC A61K 31/7125; C12N 2310/11; C12N 15/113

(57) ABSTRACT

The present disclosure relates to compositions of Mothers Against Decapentaplegic Homolog 7 (SMAD7) antisense nucleotides and methods of using the composition in treating, preventing, and/or ameliorating a skin inflammation, e.g., psoriasis, or symptoms thereof.

### CROSS-REFERENCE TO RELATED APPLICATIONS

This application claims the benefit of and priority to U.S. Provisional Patent Application No. 62/440,779, filed Dec. 30, 2016, the entire contents of which are incorporated by reference herein for all purposes.

### SEQUENCE LISTING

The instant application contains a Sequence Listing which has been submitted electronically in ASCII format and is hereby incorporated by reference in its entirety. Said ASCII copy, created on Dec. 15, 2017, is named GIU-056PC_SL.txt and is 3,855 bytes in size.

### FIELD OF THE DISCLOSURE

The present disclosure relates to compositions of Mothers Against Decapentaplegic Homolog 7 (SMAD7) antisense oligonucleotides and methods of using the compositions in treating, preventing, and/or ameliorating a skin inflammation, e.g., psoriasis, or symptoms thereof.

### BACKGROUND

Psoriasis vulgaris is recognized as the most common autoimmune disease caused by the inappropriate activation of the cellular immune system. It affects approximately 7.5 million Americans and 125 million people worldwide. While it affects people of all ages, disease onset is commonly between the ages of 15-25. Up to 30% of people with psoriasis will also develop psoriatic arthritis. Total burden of cost, direct and indirect, is estimated to be $11.25 billion annually with 40% due to work loss (reports of up to 26 missed days of work per year). Psoriasis is a common skin disorder characterized by focal formation of inflamed, raised plaques that shed scales from excessive growth of epithelial cells and involves one or more of the following histological changes in the skin:

hyperplasia of epidermal keratinocytes

vascular hyperplasia and ectasia

infiltration of T lymphocytes, neutrophils, and other types of leukocytes in the affected skin.

There remains a need for improved therapy of psoriasis. To that end, it is desirable to provide improved formulations of therapeutic agents for treating psoriasis and/or related inflammation and symptoms thereof. The present disclosure provides an improved therapy for treating psoriasis and/or related inflammation and symptoms thereof.

### SUMMARY

Provided herein, inter alia, are formulations of Mothers against decapentaplegic homolog 7 (SMAD7) antisense oligonucleotides or pharmaceutically acceptable salts thereof, and methods that are generally useful for treating, preventing, and managing skin inflammation, psoriasis, psoriatic lesions and/or psoriasis-like skin inflammation with formulations of SMAD7 antisense oligonucleotides or pharmaceutically acceptable salts thereof.

The disclosure provides novel methods for treating skin inflammation, psoriasis, psoriatic-like lesions, and/or symptoms thereof via inhibition of SMAD7, leveraging the role of SMAD7 as a key antagonist of the TGF-β signaling pathway. While other potential targets for therapeutic intervention in skin inflammation, psoriasis, psoriatic-like lesions, and/or symptoms thereof have been proposed, the present disclosure provides a new treatment shown to prevent, retard, stop, or reverse skin inflammation, psoriasis, psoriatic-like lesions, and/or symptoms thereof.

“Antisense oligonucleotide,” (“AS”) as used herein, refers to a short synthetic oligonucleotide sequence complementary to the messenger RNA (mRNA) that encodes the target protein (e.g., SMAD7). Without being bound to a particular theory, antisense oligonucleotide sequences can hybridize to a complementary region in an mRNA molecule thereby producing a double-stranded hybrid that can lead to the activation of ubiquitous catalytic enzymes, such as RNase H, which degrade DNA/RNA hybrid strands thus preventing protein translation. Without being bound by theory, an antisense oligonucleotide provided herein can hybridize to its target sequence as RNA or DNA. Thus, even if a DNA sequence is provided as target, the corresponding RNA sequence (including uracil instead of thymine) is included.

The present disclosure also provides for methods of treating skin inflammation, psoriasis, psoriatic-like lesions, and/or symptoms thereof via administering specific inhibitors of SMAD7. A “specific inhibitor,” as used herein, refers to an agent that has structural and/or functional properties that allow it to exclusively or with a high degree of selectivity act upon a molecular target. Thus, a specific inhibitor of SMAD7 possesses the inherent functional property of targeting the SMAD7 gene, its RNA or protein products, or another molecular entity whose activity or expression impinges upon the activity or expression of SMAD7 or its products either exclusively or with a high degree of specificity. Antisense oligonucleotides can be designed such that the targeting portion of the incorporated nucleotide sequence of each antisense oligonucleotide is completely or almost completely complementary to the SMAD7 mRNA sequence. Incorporation of such complementary or nearly complementary nucleotide sequences allows one to engineer antisense oligonucleotides with a high degree of specificity for a given target. Specificity can be assessed via measurement of parameters such as dissociation constants, or other criteria such as changes in protein or RNA expression levels or other assays that measure SMAD7 activity or expression.

The present disclosure also provides a method for treating skin inflammation, psoriasis, psoriatic-like lesions, and/or symptoms thereof via administration of a pharmaceutical composition including a SMAD7 antisense oligonucleotide. In another aspect, the disclosure provides a pharmaceutical composition for use in treating skin inflammation, psoriasis, psoriatic-like lesions, and/or symptoms thereof. The pharmaceutical composition may include an inhibitor of SMAD7, such as an antisense oligonucleotide that targets SMAD7, and a pharmaceutically acceptable carrier. As used herein the term “pharmaceutical composition” means, for example, a mixture containing a specified amount of a therapeutic compound, e.g., a therapeutically effective amount, of a therapeutic compound in a pharmaceutically acceptable carrier to be administered to a mammal, e.g., a human, in order to treat skin inflammation, psoriasis, psoriatic-like lesions, and/or symptoms thereof. In embodiments contemplated herein are pharmaceutical compositions including a contemplated SMAD7 antisense oligonucleotide and a pharmaceutically acceptable carrier. In another aspect, the disclosure discloses the use of a SMAD7 antisense oligonucleotide in the manufacture of a medicament for treating skin inflammation, psoriasis, psoriatic-like lesions, and/or symptoms thereof. “Medicament,” as used herein, has essentially the same meaning as the term “pharmaceutical composition.”

As used herein, “SMAD7” (also known as CRCS3, FLJ16482, MADH7, MADH8, MAD (mothers against decapentaplegic, Drosophila) homolog 7, MAD homolog 8, SMAD, mothers against DPP homolog 7, mothers against DPP homolog 8) means the human protein or any of the mRNA transcripts encoded by the gene identified by Entrez GeneID No. 4092 and allelic variants thereof.

As used herein, “SMAD7 antisense oligonucleotide” is understood to refer to an oligonucleotide comprising a nucleic acid sequence that is complementary to a nucleic acid sequence in an mRNA molecule transcribed from the SMAD7 gene. More specifically, such an oligonucleotide can be complementary to the nucleic acid sequence in the coding region of such an mRNA. In some embodiments, an SMAD7 antisense oligonucleotide can reduce the expression of SMAD7 when introduced into a cell (e.g., an immune cell, such as PBMC, pDC, or B-cell). In some embodiments, an SMAD7 antisense oligonucleotide can reduce expression of an mRNA transcribed from the gene. In some embodiments, an SMAD7 antisense oligonucleotide can reduce expression of a protein encoded by the gene. In some embodiments, an SMAD7 antisense oligonucleotide can reduce secretion of a protein encoded by the gene from the cell into which the SMAD7 antisense oligonucleotide was introduced.

Antisense oligonucleotides are short synthetic oligonucleotide sequences complementary to the mRNA, which encodes for the target protein (e.g., SMAD7). Antisense oligonucleotide sequences hybridize to the mRNA producing a double-stranded hybrid that can lead to the activation of catalytic enzymes, such as RNase H, which degrade DNA/RNA hybrid strands thus preventing protein translation.

An antisense oligonucleotide or a pharmaceutically acceptable salt thereof of the present disclosure is or may be derived from a SMAD7 antisense oligonucleotide including a sequence that is 90% to 100% identical to the sequence of 5′-GTXGCCCCTTCTCCCXGCAGC-3′ (SEQ ID NO: 1), in which X is 5-methyl 2′-deoxycytidine. In embodiments, the SMAD7 antisense oligonucleotide includes a sequence that is 90%, 95%, or 100% identical to the sequence of SEQ ID NO: 1.

An antisense oligonucleotide or a pharmaceutically acceptable salt thereof of the present disclosure is or may be derived from a SMAD7 antisense oligonucleotide including a sequence that is 90% to 100% identical to the sequence of 5′-GTXGCCCCTTCTCTCXGCAGC-3′ (SEQ ID NO: 2), in which X is 5-methyl 2′-deoxycytidine. In embodiments, the SMAD7 antisense oligonucleotide includes a sequence that is 90%, 95%, or 100% identical to the sequence of SEQ ID NO: 2.

In embodiments, the antisense oligonucleotide is an antisense oligonucleotide including SEQ ID NO: 1 or SEQ ID NO: 2, in which one or more of the internucleoside linkages is an O,O-linked phosphorothioate linkage (i.e., a phosphorothioate linkage). In embodiments, the antisense oligonucleotide is an antisense oligonucleotide including SEQ ID NO: 1 or SEQ ID NO: 2, in which all internucleoside linkages are O,O-linked phosphorothioate linkages (i.e., phosphorothioate linkages).

It is contemplated that an antisense oligonucleotide targeting SMAD7 may include a mixed-backbone in which the cytosine residues in a CpG pair are replaced by 5-methylcytosine (abbreviated as Me-dC). Methylphosphonate linkages may also be placed at the 5′ and/or 3′ ends of an antisense oligonucleotide (abbreviated as MeP).

Other exemplary antisense oligonucleotides that target SMAD7 include, but are not limited to, 5′-GTXYCCCCTTCTCCCXYCAG-3′ (SEQ ID NO: 3), in which X is a nucleotide including a nitrogenous base selected from the group consisting of cytosine and 5-methylcytosine or a 2′-O-methylcytosine nucleoside, and in which Y is a nucleotide including a nitrogenous base, e.g., guanine, 5-methylguanine, or a 2′-O-methylguanine nucleoside, provided that at least one of the nucleotides X or Y includes a methylated nitrogenous base; e.g., 5′-GTXGCCCCTTCTCCCXGCAG-3′ (SEQ ID NO: 4), in which X is 5-methyl 2′-deoxycytidine.

Contemplated antisense oligonucleotides include those that include the sequences 5′-GTC* GCC CCT TCT CCC C*YC AGC-3′ (SEQ ID NO: 5) and 5′-GTC* GCC CCT TCT CTC C*YC AGC-3′ (SEQ ID NO: 6), where C* represents 5-methyl-2′-deoxycytidine, and in which Y is a nucleotide including a nitrogenous base, e.g., guanine, 5-methylguanine, or a 2′-O-methylguanine nucleoside. In embodiments, at least one of the internucleoside linkages of a contemplated antisense oligonucleotide is an O,O-linked phosphorothioate (i.e., a phosphorothioate linkage). For example, each of the 20 internucleoside linkages of the antisense oligonucleotide of SEQ ID NO: 5 may be an O,O-linked phosphorothioate linkage.

In some embodiments, the antisense oligonucleotide is an antisense oligonucleotide including SEQ ID NO: 5 or SEQ ID NO: 6, in which one or more of the internucleoside linkages is an O,O-linked phosphorothioate linkage. In some embodiments, the antisense oligonucleotide is an antisense oligonucleotide including SEQ ID NO: 5 or SEQ ID NO: 6, in which all internucleoside linkages are O,O-linked phosphorothioate linkages.

In some embodiments, contemplated compositions disclosed herein may include a pharmaceutically acceptable salt, e.g., a sodium salt of the antisense oligonucleotide of SEQ ID NO: 1, SEQ ID NO: 2, SEQ ID NO: 3, SEQ ID NO: 4, SEQ ID NO: 5, or SEQ ID NO: 6, that optionally may include 1 to 20 O,O-linked phosphorothioate internucleoside linkages (i.e., phosphorothioate bonds). In a particular embodiment, the contemplated antisense oligonucleotide is an antisense oligonucleotide comprising the free acid form, the salt form, or the anionic form without a counterion of SEQ ID NO: 1, SEQ ID NO: 2, SEQ ID NO: 3, SEQ ID NO: 4, SEQ ID NO: 5, or SEQ ID NO: 6, wherein each of the 20 internucleoside linkages is an O,O-linked phosphorothioate linkage. In some embodiments, the phosphorothioate backbone of SEQ ID NO: 1, SEQ ID NO: 2, SEQ ID NO: 3, SEQ ID NO: 4, SEQ ID NO: 5, or SEQ ID NO: 6, can be fully or partially protonated to form an acidic form of SEQ ID NO: 1, SEQ ID NO: 2, SEQ ID NO: 3, SEQ ID NO: 4, SEQ ID NO: 5, or SEQ ID NO: 6. Contemplated salts of oligonucleotides include those that are fully neutralized, e.g., each phosphorothioate linkage is associated with an ion such as Na⁺. In some embodiments the salt of the antisense oligonucleotide of SEQ ID NO: 1, SEQ ID NO: 2, SEQ ID NO: 3, SEQ ID NO: 4, SEQ ID NO: 5, or SEQ ID NO: 6 is only partially neutralized, e.g., less than all phosphorothioate linkages are associated with an ion (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 3%, or less than 1% are neutralized). Oligonucleotides may include naturally occurring nucleobases, sugars, and covalent internucleoside (backbone) linkages as well as non-naturally occurring portions. In varying embodiments, the antisense oligonucleotides of the present disclosure, for example, the antisense oligonucleotide of SEQ ID NO: 1, SEQ ID NO: 2, SEQ ID NO: 3, SEQ ID NO: 4, SEQ ID NO: 5, or SEQ ID NO: 6, include or may include nucleotides including deoxycytidine and/or 5-methyl 2′-deoxycytidine, including, but not limited to, 5-methyl-2′-deoxycytidine 5′-monophosphate and 5-methyl-2′-deoxycytidine 5′-monophosphorothioate.

In one aspect, the present disclosure provides a method of treating, preventing, and/or ameliorating a skin inflammation or symptoms thereof in a subject in need thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition treats, prevents, and/or ameliorates the skin inflammation of the subject.

In one aspect, the present disclosure provides a method of treating, preventing, and/or ameliorating psoriasis or symptoms thereof in a subject in need thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition treats, prevents, and/or ameliorates the psoriasis or symptoms thereof of the subject.

In one aspect, the present disclosure provides a method of treating, preventing, and/or ameliorating psoriatic lesions and/or psoriasis-like skin inflammation in a subject in need thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition treats, prevents, and/or ameliorates the psoriatic lesions and/or psoriasis-like skin inflammation of the subject.

In one aspect, the present disclosure provides a method of reducing epidermal hyperproliferation of keratinocytes in a subject in need thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition reduces epidermal hyperproliferation of keratinocytes of the subject.

In one aspect, the present disclosure provides a method of reducing skin thickness in a subject in need thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition reduces skin thickness of the subject.

In one aspect, the present disclosure provides a method of maintaining remission of a skin inflammation and/or symptoms thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition maintains remission of the skin inflammation and/or symptoms thereof of the subject.

In one aspect, the present disclosure provides a method of slowing the progression of psoriatic arthritis and/or symptoms thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition slows the progression of psoriatic arthritis and/or symptoms thereof of the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to understand the invention and to demonstrate how it may be carried out in practice, embodiments are now described, by way of non-limiting example only, with reference to the accompanying drawings in which:

FIG. 1 shows images of immune-staining of SMAD7 (panels on the right) in skin specimens taken from psoriatic area of patients with psoriasis (PSO) and healthy area of control subjects (CTR). Staining with isotype control IgG is also shown (panels on the left).

FIGS. 2A-2C are images of Western blots (FIGS. 2A and 2B) and a bar graph (FIG. 2C), which show that SMAD7 sustains keratinocyte proliferation. FIG. 2A is a Western blot showing dose-dependent reduction of SMAD7 protein expression in HaCaT cells treated with increasing doses of SMAD7 antisense (AS) oligonucleotide. FIG. 2B is a Western blot of HaCaT cells treated with SMAD7 antisense oligonucleotide (AS) (20 μg/ml ). FIG. 2C is a bar graph summarizing the effects of SMAD7 knockdown on HaCaT proliferation. Data are expressed as fold-increase over control (LIPO) and indicate mean±standard deviation (SD) of 3 separate experiments (SMAD7 S-transfected cells versus SMAD7 AS-transfected cells, *P<0.01).

FIGS. 3A and 3B show a histogram and a Western blot, respectively, of effects of SMAD7 knockdown on induction of HaCaT cells to arrest in S-phase of the cell cycle. FIG. 3A is a histogram in which the values indicate the percentages of cells in the different phases of cell cycle and indicate mean±SD of 3 separate experiments. FIG. 3B is as Western blot of effects of SMAD7 knockdown in HaCaT cells, showing enhancement of eIF2α phosphorylation and downregulation of CDC25A expression. CDC25A, CDC25B and CDC25C expression were assessed by Western blotting. One of 3 representative experiments in which similar results were obtained is shown.

FIGS. 4A-4B are Western blot images and a bar graph, respectively, showing that SMAD7 overexpression results in increase in keratin (K) 6A and 16 expression and positive regulation of HaCaT proliferation. FIG. 4A shows K6A and K16 expression by Western blotting. β-actin was used as loading control. FIG. 4B is a bar graph of effects SMAD7 AS on HaCaT cell proliferation. BrdU-positive cells were evaluated by ELISA using a commercial colorimetric assay. Data are expressed as fold-increase over control and indicate mean±SD of 3 separate experiments (*P<0.01).

FIGS. 5A-5B are Western blot and immunostaining images, respectively, which show that SMAD7 expression is increased in the skin of mice with Aldara-mediated psoriasis-like lesions. FIG. 5A is a Western blot of SMAD7 expression of mouse skin after Aldara cream was applied daily on the shaved back skin of C57BL/6 mice. FIG. 5B shows immunohistochemistry of the mouse skin as treated for FIG. 5A. The figures are representative of six separate experiments. β-actin was used as loading control in Western blotting studies. Staining with isotype control IgG is also shown in FIG. 5B.

FIGS. 6A-6D show immunostaining (FIG. 6A), a bar graph (FIG. 6B), Western blots (FIG. 6C), and immunostaining (FIG. 6D), showing that inhibition of SMAD7 with a specific SMAD7 antisense oligonucleotide reduces skin thickness and keratinocyte proliferation. Aldara cream was applied daily on the shaved back skin of C57BL/6 mice for 4 days and SMAD7 antisense (AS) or sense (S) (125 μg/mouse) oligonucleotides were topically applied each day starting 12 hours after Aldara treatment. FIG. 6A shows representative stainings with hematoxylin and eosin of skin sections of mice treated as above. One of 3 separate experiments, in which 12 mice per group were analysed, is shown. FIG. 6B shows a bar graph summarizing epidermal thickness of skin sections of mice treated as above (FIG. 6A) and data are expressed as mean±SD of all experiments. SMAD7 AS-treated mice vs SMAD7 S-treated mice P<0.01. FIG. 6C shows panels of Western blots of total proteins extracted from the skin of mice were treated as above (FIG. 6A). One representative experiment is shown. FIG. 6D shows representative immunostaining for Ki67 (right panels) of skin sections of mice treated as indicated in FIG. 6A. Staining with isotype IgG is also shown (left panels).

FIG. 7A shows representative stainings with hematoxylin and eosin of skin sections of mice treated with Aldara cream for the indicated time points. FIG. 7B shows a bar graph of K6A and K16 expression in total extracts of treated skin as indicated in FIG. 7A. FIG. 7C shows Western blots of proteins (K6A, K16, and β-actin (control)) performed to evaluate epidermal thickness in skin sections of mice treated with daily cutaneous administration of Aldara cream. Data are expressed as mean±SD of 3 separate experiments. Control mice (day 0) vs Aldara-treated mice *P<0.05. β-actin was used as loading control.

### DETAILED DESCRIPTION

Provided herein, inter alia, are formulations of Mothers against decapentaplegic homolog 7 (SMAD7) antisense oligonucleotides or pharmaceutically acceptable salts thereof, and methods that are generally useful for treating, preventing, and managing skin inflammation, psoriasis, psoriatic lesions and/or psoriasis-like skin inflammation with formulations of SMAD7 antisense oligonucleotides or pharmaceutically acceptable salts thereof. A SMAD7 antisense oligonucleotide may be an oligonucleotide that is capable of binding to a SMAD7 mRNA transcript and inducing degradation of the SMAD7 mRNA transcript, preventing splicing of the SMAD7 mRNA transcript, or preventing protein translation of the SMAD7 mRNA transcript.

### Definitions

A “patient,” as described herein, refers to any animal suffering from or diagnosed for a skin inflammation, e.g., psoriasis, including, but not limited to, mammals, primates, and humans. In certain embodiments, the patient may be a non-human mammal such as, for example, a cat, a dog, or a horse. In a preferred embodiment, the patient is a human subject.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. The terms “subject” and “patient” may be used interchangeably, however, in some embodiments a subject may not be diagnosed with or is suffering from a disease or disorder, though may be in need of therapy.

“Treating,” includes any effect, e.g., lessening, reducing, modulating, preventing, or eliminating, that results in the improvement of the condition, disease, disorder, etc. “Treating” or “treatment” of a disease state includes: (1) inhibiting the disease state, i.e., arresting the development of the disease state or its clinical symptoms; (2) relieving the disease state, i.e., causing temporary or permanent regression of the disease state or its clinical symptoms; (3) reducing or lessening the symptoms of the disease state; or (4) preventing the disease state, e.g., causing the clinical symptoms of the disease state not to develop in a subject that may be exposed to or predisposed to the disease state, but does not yet experience or display symptoms of the disease state. As used herein, “preventing” or “prevent” describe reducing or eliminating the onset of the symptoms or complications of the disease, condition or disorder. The term “preventing,” when used in relation to a condition, such as intraocular pressure, is art-recognized, and refers to formulation, composition and/or device (e.g., ocular insert) which reduces the frequency of, or delays the onset of, signs and/or symptoms of a medical condition in a subject relative to a subject which does not receive the composition.

By “reduce” or other forms of the word, such as “reducing” or “reduction,” is meant lowering of an event or characteristic (e.g., vascular leakage). It is understood that this is typically in relation to some standard or expected value, in other words it is relative, but that it is not always necessary for the standard or relative value to be referred to.

Insofar as the methods of the present disclosure are directed to preventing disorders, it is understood that the term “prevent” does not require that the disease state be completely thwarted. Rather, as used herein, the term preventing refers to the ability of the skilled artisan to identify a patient or a population that is susceptible to disorders, such that administration of the compounds of the present disclosure may occur prior to onset of a disease. The term does not imply that the disease state be completely avoided.

The term “ameliorating a symptom” or other forms of the word such as “ameliorate a symptom” is used herein to mean that administration of a therapeutic agent of the present disclosure mitigates one or more symptoms of a disease or a disorder in a host and/or reduces, inhibits, or eliminates a particular symptom associated with the disease or disorder prior to and/or post-administration of the therapeutic agent.

The terms “manage,” “management,” “managing,” and the like are used herein to generally mean controlling the severity or manifestation of symptoms of a disease, or the means of treating the disease. Generally, management is used to obtain a desired pharmacological and/or physiological effect. The effect may be therapeutic in terms of partially or completely curing a disease and/or adverse effect attributed to the disease or ensuring that a particular symptom or manifestation of the disease does not occur or reoccur in a patient or does not rise to an undesirable or intolerable level in a patient. The term “management” as used herein covers any management of a disease in a mammal, particularly a human, and includes: (a) inhibiting the disease, i.e., preventing the disease from increasing in severity or scope; (b) relieving the disease, i.e., causing partial or complete amelioration of the disease; or (c) preventing relapse of the disease, i.e., preventing the disease from returning to an active state following previous successful treatment of symptoms of the disease or treatment of the disease. “Management” as used herein may also be used with reference to administration of a specific treatment for the disease, for example, a SMAD7 antisense oligonucleotide.

“Effective amount,” as used herein, refers to the amount of an agent that is sufficient to at least partially treat a condition when administered to a patient. The therapeutically effective amount will vary depending on the condition, the route of administration of the component, and the age, weight, etc. of the patient being treated. Accordingly, an effective amount of a specific inhibitor of SMAD7 is the amount of inhibitor necessary to treat skin inflammation, psoriasis, psoriatic-like lesions, and/or symptoms thereof in a patient such that administration of the agent prevents the skin inflammation, psoriasis, psoriatic-like lesions, and/or symptoms thereof from occurring in a subject; prevents skin inflammation, psoriasis, psoriatic-like lesions, and/or symptoms thereof from progressing; or relieves or completely ameliorates the associated symptoms of the skin inflammation, psoriasis, psoriatic-like lesions, and/or symptoms thereof, i.e., causes regression of the disease.

The terminology used herein is for the purpose of describing particular embodiments only, and is not intended to limit the scope of the present disclosure. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. All percentages and ratios used herein, unless otherwise indicated, are by weight.

Unless otherwise defined, all technical and scientific terms used herein have the same meaning as commonly understood by one of ordinary skill in the art to which this disclosure belongs. In the case of conflict, the present specification will control. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed disclosure. In the case of conflict, the present specification, including definitions, will control. In addition, the materials, methods and examples are illustrative only and are not intended to be limiting.

By “pharmaceutically acceptable” is meant a material that is not biologically or otherwise undesirable, i.e., the material can be administered to an individual along with the relevant active compound without causing clinically unacceptable biological effects or interacting in a deleterious manner with any of the other components of the pharmaceutical composition in which it is contained.

As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption-delaying agents, and the like, that are compatible with pharmaceutical administration. The use of such media and agents for pharmaceutically active substances is known in the art. In certain embodiments the pharmaceutical composition is administered orally and includes an enteric coating suitable for regulating the site of absorption of the encapsulated substances within the digestive system or gut. For example, an enteric coating can include an ethylacrylate-methacrylic acid copolymer.

In certain embodiments, a contemplated SMAD7 antisense oligonucleotide and any pharmaceutical composition thereof may be administered by one or several routes, including orally, topically, parenterally, e.g., subcutaneous injection, by inhalation spray, or rectally. The term parenteral as used herein includes subcutaneous injections, intrapancreatic administration, intravenous, intramuscular, intraperitoneal, intrasternal injection or infusion techniques. For example, the SMAD7 antisense oligonucleotide may be administered subcutaneously to a subject. In another example, the SMAD7 antisense oligonucleotide may be administered orally to a subject. In another example, the SMAD7 antisense oligonucleotide may be administered directly to a site of skin inflammation, psoriasis, or psoriatic-like lesions via parenteral administration.

Throughout the description and claims of this specification the word “comprise” and other forms of the word, such as “comprising” and “comprises,” means including but not limited to, and is not intended to exclude, for example, other additives, components, integers, or steps.

“Optional” or “optionally” means that the subsequently described event or circumstance can or cannot occur, and that the description includes instances where the event or circumstance occurs and instances where it does not.

“Inhibitor,” as used herein, refers to an agent capable of decreasing expression of a gene or DNA sequence, preventing or suppressing production, activity, or translation of an RNA product of a gene into protein, or preventing or suppressing the activity of the protein product of a gene, through either a direct or indirect interaction with the gene, RNA product, or protein product of a gene or any transitional forms of these entities or another molecular entity whose activity or expression impinges upon the activity or expression of the intended target. Such inhibitors may include, but are not limited to, for example, antibodies, small molecules that bind to a specific molecular target, and antisense oligonucleotides targeted to specific mRNA transcripts. Accordingly, “inhibitor of SMAD7,” as used herein, refers to an agent capable of decreasing expression of SMAD7; preventing or suppressing production, activity, or translation of an RNA product of SMAD7 into protein; or preventing or suppressing the activity of the protein product of SMAD7, through either a direct or indirect interaction with the gene, RNA product, or protein product of SMAD7 or any transitional forms of these entities or another molecular entity whose activity or expression impinges upon the activity or expression of SMAD7.

### Methods of Treating, Preventing, and/or Ameliorating a Skin Inflammation or Symptoms

In one aspect, the present disclosure provides a method of treating, preventing, and/or ameliorating a skin inflammation or symptoms thereof in a subject in need thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition treats, prevents, and/or ameliorates the skin inflammation of the subject. In certain embodiments, the present disclosure provides a method of treating, preventing, and/or ameliorating a skin inflammation or symptoms thereof in a subject in need thereof, the method including administering to the subject a pharmaceutical composition including an effective amount of a SMAD7 antisense oligonucleotide (e.g., an antisense oligonucleotide of SEQ ID NO: 1 or 2, or a pharmaceutically acceptable salt thereof), and upon administration the composition treats, prevents, and/or ameliorates the skin inflammation of the subject. In certain embodiments, the present disclosure provides a method of treating, preventing, and/or ameliorating a skin inflammation or symptoms thereof in a subject in need thereof, the method including administering to the subject a pharmaceutical composition including an effective amount of a SMAD7 antisense oligonucleotide (e.g., an antisense oligonucleotide of SEQ ID NO: 5 or 6, or a pharmaceutically acceptable salt thereof), and upon administration the composition treats, prevents, and/or ameliorates the skin inflammation of the subject.

In one aspect, the present disclosure provides a method of treating, preventing, and/or ameliorating psoriasis or symptoms thereof in a subject in need thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition treats, prevents, and/or ameliorates the psoriasis or symptoms thereof of the subject. In certain embodiments, the present disclosure provides a method of treating, preventing, and/or ameliorating psoriasis or symptoms thereof in a subject in need thereof, the method including administering to the subject a pharmaceutical composition including an effective amount of a SMAD7 antisense oligonucleotide (e.g., an antisense oligonucleotide of SEQ ID NO: 1 or 2, or a pharmaceutically acceptable salt thereof), and upon administration the composition treats, prevents, and/or ameliorates the psoriasis or symptoms thereof, of the subject. In certain embodiments, the present disclosure provides a method of treating, preventing, and/or ameliorating psoriasis or symptoms thereof in a subject in need thereof, the method including administering to the subject a pharmaceutical composition including an effective amount of a SMAD7 antisense oligonucleotide (e.g., an antisense oligonucleotide of SEQ ID NO: 5 or 6, or a pharmaceutically acceptable salt thereof), and upon administration the composition treats, prevents, and/or ameliorates the psoriasis or symptoms thereof, of the subject.

In one aspect, the present disclosure provides a method of treating, preventing, and/or ameliorating psoriatic lesions and/or psoriasis-like skin inflammation in a subject in need thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition treats, prevents, and/or ameliorates the psoriatic lesions and/or psoriasis-like skin inflammation of the subject. In certain embodiments, the present disclosure provides a method of treating, preventing, and/or ameliorating psoriatic lesions and/or psoriatic-like skin inflammation in a subject in need thereof, the method including administering to the subject a pharmaceutical composition including an effective amount of a SMAD7 antisense oligonucleotide (e.g., an antisense oligonucleotide of SEQ ID NO: 1 or 2, or a pharmaceutically acceptable salt thereof), and upon administration the composition treats, prevents, and/or ameliorates the psoriatic-lesions or psoriatic-like skin inflammation of the subject. In certain embodiments, the present disclosure provides a method of treating, preventing, and/or ameliorating a psoriatic lesions and/or psoriatic-like skin inflammation in a subject in need thereof, the method including administering to the subject a pharmaceutical composition including an effective amount of a SMAD7 antisense oligonucleotide (e.g., an antisense oligonucleotide of SEQ ID NO: 5 or 6, or a pharmaceutically acceptable salt thereof), and upon administration the composition treats, prevents, and/or ameliorates the psoriatic-lesions or psoriatic-like skin inflammation of the subject.

### Method or Reducing Cell Hyperproliferation

In one aspect, the present disclosure provides a method of reducing epidermal hyperproliferation of keratinocytes in a subject in need thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition reduces epidermal hyperproliferation of keratinocytes of the subject. In certain embodiments, the present disclosure provides a method of reducing epidermal hyperproliferation of keratinocytes in a subject in need thereof, the method including administering to the subject a pharmaceutical composition including an effective amount of a SMAD7 antisense oligonucleotide (e.g., an antisense oligonucleotide of SEQ ID NO: 1 or 2, or a pharmaceutically acceptable salt thereof), and upon administration the composition reduces epidermal hyperproliferation of keratinocytes of the subject. In certain embodiments, the present disclosure provides a method of reducing epidermal hyperproliferation of keratinocytes in a subject in need thereof, the method including administering to the subject a pharmaceutical composition including an effective amount of a SMAD7 antisense oligonucleotide (e.g., an antisense oligonucleotide of SEQ ID NO: 5 or 6, or a pharmaceutically acceptable salt thereof), and upon administration the composition reduces epidermal hyperproliferation of keratinocytes of the subject.

In one aspect, the present disclosure provides a method of reducing skin thickness in a subject in need thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition reduces skin thickness of the subject. In certain embodiments, the present disclosure provides a method of reducing skin thickness in a subject in need thereof, the method including administering to the subject a pharmaceutical composition including an effective amount of a SMAD7 antisense oligonucleotide (e.g., an antisense oligonucleotide of SEQ ID NO: 1 or 2, or a pharmaceutically acceptable salt thereof), and upon administration the composition reduces skin thickness of the subject. In certain embodiments, the present disclosure provides a method of reducing skin thickness in a subject in need thereof, the method including administering to the subject a pharmaceutical composition including an effective amount of a SMAD7 antisense oligonucleotide (e.g., an antisense oligonucleotide of SEQ ID NO: 5 or 6, or a pharmaceutically acceptable salt thereof), and upon administration the composition reduces skin thickness of the subject.

### Method of Maintaining Remission of Inflammation

In one aspect, the present disclosure provides a method of maintaining remission of a skin inflammation and/or symptoms thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition maintains remission of the skin inflammation and/or symptoms thereof of the subject. In certain embodiments, the present disclosure provides a method of maintaining remission of the skin inflammation and/or symptoms thereof in a subject in need thereof, the method including administering to the subject a pharmaceutical composition including an effective amount of a SMAD7 antisense oligonucleotide (e.g., an antisense oligonucleotide of SEQ ID NO: 1 or 2, or a pharmaceutically acceptable salt thereof), and upon administration the composition maintains remission of the skin inflammation and/or symptoms thereof of the subject. In certain embodiments, the present disclosure provides a method of maintaining remission of the skin inflammation and/or symptoms thereof in a subject in need thereof, the method including administering to the subject a pharmaceutical composition including an effective amount of a SMAD7 antisense oligonucleotide (e.g., an antisense oligonucleotide of SEQ ID NO: 5 or 6, or a pharmaceutically acceptable salt thereof), and upon administration the composition maintains remission of the skin inflammation and/or symptoms thereof of the subject.

### Method of Slowing Progression of Disease and/or Symptoms

In one aspect, the present disclosure provides a method of slowing the progression of psoriatic arthritis and/or symptoms thereof, the method including administering to the subject a pharmaceutical composition including an effective amount of a SMAD7 antisense oligonucleotide (e.g., an antisense oligonucleotide of SEQ ID NO: 1, 2, 3, 4, 5, or 6, or a pharmaceutically acceptable salt thereof), and upon administration the composition slows the progression of psoriatic arthritis and/or symptoms thereof of the subject. In certain embodiments, the present disclosure provides a method of slowing the progression of psoriatic arthritis and/or symptoms thereof in a subject in need thereof, the method including administering to the subject a pharmaceutical composition including an effective amount of a SMAD7 antisense oligonucleotide (e.g., an antisense oligonucleotide of SEQ ID NO: 1 or 2, or a pharmaceutically acceptable salt thereof), and upon administration the composition slows the progression of psoriatic arthritis and/or symptoms thereof of the subject. In certain embodiments, the present disclosure provides a method of slowing the progression of psoriatic arthritis and/or symptoms thereof in a subject in need thereof, the method including administering to the subject a pharmaceutical composition including an effective amount of a SMAD7 antisense oligonucleotide (e.g., an antisense oligonucleotide of SEQ ID NO: 5 or 6, or a pharmaceutically acceptable salt thereof), and upon administration the composition slows the progression of psoriatic arthritis and/or symptoms thereof of the subject.

### Dosage Forms of SMAD7 Antisense Oligonucleotides as SMAD7 Antisense Oligonucleotide

In one aspect, the present disclosure provides formulations including a SMAD7 antisense oligonucleotide (e.g., an antisense oligonucleotide of SEQ ID NO: 1, 2, 3, 4, 5, or 6) or a pharmaceutically acceptable salt thereof for use in therapy, e.g., treating, preventing, and/or ameliorating a skin inflammation. In certain embodiments, the formulation is used for treating, preventing and/or ameliorating a skin inflammation such as a psoriasis-like lesion, a psoriatic lesion, or psoriasis. In certain embodiments, the formulation is used for treating, preventing and/or ameliorating a pediatric skin inflammation.

Pharmaceutical compositions containing a SMAD7 antisense oligonucleotide, such as those disclosed herein, may be presented in a dosage unit form and may be prepared by any suitable method. A pharmaceutical composition should be formulated to be compatible with its intended route of administration. Useful formulations can be prepared by methods well known in the pharmaceutical arts. For example, see Remington's Pharmaceutical Sciences, 18th ed. (Mack Publishing Company, 1990).

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

In some embodiments contemplated herein are compositions suitable for oral delivery (e.g., capsules, tablets, caplets, pills, troches, lozenges, powders, and granules) of an antisense oligonucleotide. Contemplated SMAD7 antisense oligonucleotides include oligonucleotides (e.g., antisense oligonucleotides of SEQ ID NO: 1, 2, 3, 4, 5, or 6, or pharmaceutically acceptable salts thereof) that act against SMAD7 and may be administered orally. Disclosed therapies may, when administered orally to a subject suffering from a skin inflammation, e.g., psoriasis, deliver an effective amount of an antisense oligonucleotide to the intestinal system of a patient, e.g., deliver an effective amount of an antisense oligonucleotide to the terminal ileum and/or right colon of a patient.

The formulation of a SMAD7 antisense oligonucleotide (e.g., an antisense oligonucleotide of SEQ ID NO: 1, 2, 3, 4, 5, or 6, or a pharmaceutically acceptable salt thereof) may be an oral pharmaceutical composition. In embodiments, the oral pharmaceutical composition includes a SMAD7 antisense oligonucleotide (e.g., SEQ ID NO: 1 or 2, or a pharmaceutically acceptable salt thereof). In embodiments, the oral pharmaceutical composition includes a SMAD7 antisense oligonucleotide (e.g., an antisense oligonucleotide of SEQ ID NO: 5 or 6, or a pharmaceutically acceptable salt thereof).

The pharmaceutical formulation of a SMAD7 antisense oligonucleotide (e.g., an antisense oligonucleotide of SEQ ID NO: 1, 2, 3, 4, 5, or 6, or a pharmaceutically acceptable salt thereof) may be a tablet or capsule.

The tablet of a SMAD7 antisense oligonucleotide (e.g., an antisense oligonucleotide of SEQ ID NO: 1, 2, 3, 4, 5, or 6, or a pharmaceutically acceptable salt thereof) may be formulated as a minitablet or a microtablet. The tablet of a SMAD7 antisense oligonucleotide (e.g., an antisense oligonucleotide of SEQ ID NO: 1, 2, 3, 4, 5, or 6, or a pharmaceutically acceptable salt thereof) may include minitablets, microtablets, or granulates. The capsule of a SMAD7 antisense oligonucleotide (e.g., an antisense oligonucleotide of SEQ ID NO: 1, 2, 3, 4, 5, or 6, or a pharmaceutically acceptable salt thereof) may include minitablets, microtablets, or granulates. The oral pharmaceutical composition may be enteric-coated. In certain embodiments, the oral pharmaceutical composition (tablet or capsule) of a SMAD7 antisense oligonucleotide (e.g., an antisense oligonucleotide of SEQ ID NO: 1 or 2, or a pharmaceutically acceptable salt thereof) is enteric-coated or includes enteric-coated micropellets, microtablets, minitablets, or granulates. In certain embodiments, the oral pharmaceutical composition (tablet or capsule) of a SMAD7 antisense oligonucleotide (e.g., SEQ ID NO: 5 or 6, or a pharmaceutically acceptable salt thereof) is enteric-coated or includes enteric-coated micropellets, microtablets, minitablets, or granulates.

In certain embodiments, the oral pharmaceutical composition of a SMAD7 antisense oligonucleotide (e.g., an antisense oligonucleotide of SEQ ID NO: 1, 2, 3, 4, 5, or 6, or a pharmaceutically acceptable salt thereof) is not enteric-coated. In certain embodiments, the oral pharmaceutical composition of a SMAD7 antisense oligonucleotide (e.g., an antisense oligonucleotide of SEQ ID NO: 1 or 2, or a pharmaceutically acceptable salt thereof) is not enteric-coated. In certain embodiments, the oral pharmaceutical composition of a SMAD7 antisense oligonucleotide (e.g., an antisense oligonucleotide of SEQ ID NO: 5 or 6, or a pharmaceutically acceptable salt thereof) is not enteric-coated.

In embodiments, the compositions of a SMAD7 antisense oligonucleotide (i.e., a therapy including a SMAD7 antisense oligonucleotide (e.g., an antisense oligonucleotide of SEQ ID NO: 1, 2, 3, 4, 5, or 6, or a pharmaceutically acceptable salt thereof)) may be suitable for oral delivery of an antisense oligonucleotide, e.g., tablets, that include an enteric coating, e.g., a gastro-resistant coating, such that the compositions may deliver the antisense compound to, e.g., the terminal ileum and right colon of a patient. Such administration may result in a topical effect, for example, by substantially topically applying the antisense compound directly to an affected portion of the intestine of a subject. Such administration, may, in embodiments, substantially avoid unwanted systemic absorption of the antisense compound.

For example, an oral dosage form (e.g., tablet) for oral administration may include granules (e.g., an oral dosage form at least partially formed from granules) that include a disclosed antisense compound (e.g., an antisense oligonucleotide of SEQ ID NO: 1, 2, 3, 4, 5, or 6, or a pharmaceutically acceptable salt thereof) and pharmaceutically acceptable excipients. Such an oral dosage form, e.g., a tablet, may be coated with an enteric coating. Contemplated oral dosage forms, e.g., tablets, may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In embodiments, contemplated oral dosage forms of the pharmaceutical formulations include an intra-granular phase that includes a contemplated antisense oligonucleotide (e.g., an antisense oligonucleotide of SEQ ID NO: 1, 2, 3, 4, 5, or 6, or a pharmaceutically acceptable salt thereof) and a pharmaceutically acceptable filler. For example, a SMAD7 antisense oligonucleotide (e.g., an antisense oligonucleotide of SEQ ID NO: 1, 2, 3, 4, 5, or 6, or a pharmaceutically acceptable salt thereof) and a filler may be blended together, with optionally other excipients, and formed into granules. In embodiments, the intragranular phase may be formed using wet granulation, e.g., a liquid (e.g., water) is added to the blended antisense compound and filler, and then the combination is dried, milled and/or sieved to produce granules. Other processes in the art may be used to achieve an intragranular phase.

In embodiments, contemplated oral dosage forms of the formulations include an extra-granular phase, which may include one or more pharmaceutically acceptable excipients, and which may be blended with the intragranular phase to form a disclosed formulation.

A SMAD7 antisense oligonucleotide (e.g., an antisense oligonucleotide of SEQ ID NO: 1, 2, 3, 4, 5, or 6, or a pharmaceutically acceptable salt thereof) formulation (e.g., oral dosage form) may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pregelatinized starch, calcium carbonate, and others including combinations thereof.

In embodiments, a SMAD7 antisense oligonucleotide (e.g., an antisense oligonucleotide of SEQ ID NO: 1, 2, 3, 4, 5, or 6, or a pharmaceutically acceptable salt thereof) formulation (e.g., oral dosage form) may include an intragranular phase and/or an extragranular phase that includes a binder, which may generally function to hold the ingredients of the pharmaceutical formulation together. Exemplary binders include, but are not limited to, the following: starches, sugars, cellulose or modified cellulose such as hydroxypropyl cellulose, lactose, pregelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated SMAD7 antisense oligonucleotide (e.g., an antisense oligonucleotide of SEQ ID NO: 1, 2, 3, 4, 5, or 6, or a pharmaceutically acceptable salt thereof) formulations (e.g., oral dosage form), e.g., that include an intragranular phase and/or an extragranular phase, may include a disintegrant such as but not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In certain embodiments, a contemplated SMAD7 antisense oligonucleotide (e.g., an antisense oligonucleotide of SEQ ID NO: 1, 2, 3, 4, 5, or 6, or a pharmaceutically acceptable salt thereof) formulation includes an intra-granular phase including a disclosed antisense oligonucleotide and excipients chosen from: mannitol, microcrystalline cellulose, hydroxypropylmethyl cellulose, and sodium starch glycolate or combinations thereof, and an extra-granular phase including one or more of: microcrystalline cellulose, sodium starch glycolate, and magnesium stearate or mixtures thereof.

In certain embodiments, a contemplated SMAD7 antisense oligonucleotide (e.g., an antisense oligonucleotide of SEQ ID NO: 1, 2, 3, 4, 5, or 6, or a pharmaceutically acceptable salt thereof) formulation may include a lubricant, e.g., an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In certain embodiments, pharmaceutical formulations of the present disclosure include an enteric coating. Generally, enteric coatings create a barrier for the oral medication that controls the location at which the drug is absorbed along the digestive tract. Enteric coatings may include a polymer that disintegrates at different rates according to pH. Enteric coatings may include, for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

In certain embodiments, the enteric coating includes an anionic, cationic, or neutral copolymer based on methacrylic acid, methacrylic/acrylic esters or their derivatives. In certain embodiments, the enteric coating includes an ethylacrylate-methacrylic acid copolymer. Commercially available enteric coatings include Opadry® AMB, ethylacrylate-methacrylic acid copolymers (e.g., Acryl-EZE®), dimethylaminoethyl methacrylate-butyl methacrylate-methyl methacrylate copolymer (2:1:1), or poly(methacrylic acid-co-methyl-methacrylate) 1:1 and poly(methacrylic acid-co-methyl-methacrylate) 1:2 copolymers (e.g., Eudragit®) grades. In embodiments, the enteric coating makes up about 5% to about 10%, about 5% to about 20%, about 8 to about 15%, about 8% to about 18%, about 10% to about 12%, or about 12% to about 16%, of a contemplated tablet by weight.

For example, a SMAD7 antisense oligonucleotide (e.g., an antisense oligonucleotide of SEQ ID NO: 1, 2, 3, 4, 5, or 6, or a pharmaceutically acceptable salt thereof) in the form of a tablet is provided that includes about 0.5% to about 70%, e.g., about 0.5% to about 10%, or about 1% to about 20%, by weight of an antisense oligonucleotide or a pharmaceutically acceptable salt thereof. Such a tablet may include for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose. For example, a contemplated tablet may include an intragranular phase that includes about 30% to about 60%, about 45% to about 65% by weight, or alternatively, about 5% to about 10% by weight of an antisense oligonucleotide of SEQ ID NO: 1, 2, 3, 4, 5, or 6, or a pharmaceutically acceptable salt thereof, about 30% to about 50%, or alternatively, about 5% to about 15% by weight mannitol, about 5% to about 15% microcrystalline cellulose, about 0% to about 4%, or about 1% to about 7% hydroxypropylmethyl cellulose, and about 0% to about 4%, e.g., about 2% to about 4% sodium starch glycolate by weight.

Exemplary SMAD7 antisense oligonucleotide formulations include dosage forms that include or consist essentially of about 10 mg to about 500 mg of an antisense oligonucleotide of SEQ ID NO: 1, 2, 3, 4, 5, or 6, or a pharmaceutically acceptable salt thereof, for example, tablets that include about 10 mg, about 15 mg, about 20 mg, about 25 mg, about 30 mg, about 35 mg, about 40 mg, about 50 mg, about 60 mg, about 70 mg, about 80 mg, about 90 mg, about 100 mg, about 150 mg, about 200 mg, or about 250 mg of an antisense oligonucleotide of SEQ ID NO: 1, 2, 3, 4, 5, or 6, or a pharmaceutically acceptable salt thereof, are contemplated herein. In certain embodiments, the SMAD7 antisense oligonucleotide (e.g., an antisense oligonucleotide of SEQ ID NO: 1, 2, 3, 4, 5, or 6, or a pharmaceutically acceptable salt thereof) may be a tablet for oral use including: about 0.5% to about 10% by weight of a SMAD7 antisense oligonucleotide; about 30% to about 50% by weight mannitol; and about 10% to about 30% by weight microcrystalline cellulose.

In an exemplary embodiment of the disclosure, a pharmaceutically acceptable tablet for oral administration is provided that includes an intra-granular phase that may include about 50% by weight of an antisense oligonucleotide of SEQ ID NO: 1, 2, 3, 4, 5, or 6, or a pharmaceutically acceptable salt thereof, about 11.5% by weight mannitol, about 10% by weight microcrystalline cellulose, about 3% by weight hydroxypropylmethyl cellulose, and about 2.5% by weight sodium starch glycolate; and an extra-granular phase that may include about 20% by weight microcrystalline cellulose, about 2.5% by weight sodium starch glycolate, and about 0.5% by weight magnesium stearate. The tablet may also include an enteric coating.

In another exemplary embodiment, a pharmaceutically acceptable tablet for oral administration is provided that includes or consists essentially of: an intra-granular phase that may include about 5% to about 10%, e.g., about 8% by weight of an antisense oligonucleotide of SEQ ID NO: 1, 2, 3, 4, 5, or 6, or a pharmaceutically acceptable salt thereof (e.g., a sodium salt), about 40% by weight mannitol, about 8% by weight microcrystalline cellulose, about 5% by weight hydroxypropylmethyl cellulose, and about 2% by weight sodium starch glycolate; and an extra-granular phase that may include about 17% by weight microcrystalline cellulose, about 2% by weight sodium starch glycolate, and about 0.4% by weight magnesium stearate.

Contemplated tablets may also include an enteric coating, e.g., a disclosed tablet may include about 13%, about 14%, about 15%, about 16%, or about 17% by weight of an enteric coating, e.g., ethylacrylate-methacrylic acid copolymers (e.g., AcrylEZE®).

For example, the SMAD7 antisense oligonucleotide may be in the form of a pharmaceutically acceptable tablet for oral use including an intra-granular phase and extra-granular phase, in which for example, the intra-granular phase includes about 5% to about 10%, by weight (for example about 8% by weight) of an antisense oligonucleotide represented by SEQ ID NO: 4 or a pharmaceutically acceptable salt thereof, about 40% by weight mannitol, about 8% by weight microcrystalline cellulose, about 5% by weight hydroxypropylmethyl cellulose, and about 2% by weight sodium starch glycolate, and for example, the extra-granular phase includes about 17% by weight microcrystalline cellulose, about 2% by weight sodium starch glycolate, and about 0.4% by weight magnesium stearate, where the tablet may further include an enteric coating.

Contemplated formulations, e.g., tablets, in embodiments, when orally administered to the patient may result in minimal plasma concentration of the antisense oligonucleotide in the patient. In another embodiment, contemplated formulations, when orally administered to a patient, topically deliver to the terminal ileum and/or right colon of a patient, e.g., to an affected or diseased intestinal site of a patient. Formulations suitable for oral administration may be in the form of capsules, cachets, pills, tablets, lozenges (using, e.g., a flavored basis such as sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present disclosure may also be administered as a bolus, electuary, or paste.

In certain embodiments, a pharmaceutical oral dosage form (e.g., tablet formulation) of a SMAD7 antisense oligonucleotide may include an intra-granular phase, where the intra-granular phase includes an antisense oligonucleotide such as an antisense oligonucleotide of SEQ ID NO: 1, 2, 3, 4, 5, or 6, or a pharmaceutically acceptable salt thereof (e.g., a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 16% by weight of the tablet. For example, a pharmaceutically acceptable tablet for oral use may include about 0.5% to about 10% by weight of an antisense oligonucleotide, e.g., an antisense oligonucleotide of SEQ ID NO: 1, 2, 3, 4, 5, or 6, or a pharmaceutically acceptable salt thereof, about 30% to 50% by weight mannitol, about 10% to 30% by weight microcrystalline cellulose, and an enteric coating including an ethylacrylate-methacrylic acid copolymer.

In another example, a pharmaceutically acceptable tablet for oral use may include an intra-granular phase, including about 5% to about 10% by weight of an antisense oligonucleotide, e.g., an antisense oligonucleotide of SEQ ID NO: 1, 2, 3, 4, 5, or 6, or a pharmaceutically acceptable salt thereof, about 40% by weight mannitol, about 8% by weight microcrystalline cellulose, about 5% by weight hydropropylmethyl cellulose, and about 2% by weight sodium starch glycolate; an extra-granular phase including about 17% by weight microcrystalline cellulose, about 2% by weight sodium starch glycolate, about 0.4% by weight magnesium stearate; and an enteric coating over the tablet including an ethylacrylate-methacrylic acid copolymer.

In some embodiments the pharmaceutical composition may contain an enteric coating including about 13%, about 15%, about 16%, about 17% or about 18% by weight, e.g., AcyrlEZEO (see, e.g., PCT Publication No. WO2010/054826, which is hereby incorporated by reference in its entirety).

The rate at which point the coating dissolves and the active ingredient is released is its dissolution rate. In an embodiment, a contemplated tablet may have a dissolution profile, e.g., when tested in a USP/EP Type 2 apparatus (paddle) at 100 rpm and 37° C. in a phosphate buffer with a pH of 7.2, of about 50% to about 100% of the oligonucleotide releasing after about 120 minutes to about 240 minutes, for example after 180 minutes. In another embodiment, a contemplated tablet may have a dissolution profile, e.g., when tested in a USP/EP Type 2 apparatus (paddle) at 100 rpm and 37° C. in diluted HCl with a pH of 1.0, where substantially none of the oligonucleotide is released after 120 minutes. A contemplated tablet, in another embodiment, may have a dissolution profile, e.g. when tested in USP/EP Type 2 apparatus (paddle) at 100 rpm and 37° C. in a phosphate buffer with a pH of 6.6, of about 10% to about 30%, or not more than about 50%, of the oligonucleotide releasing after 30 minutes.

Disclosed formulations, e.g. tablets, in embodiments, when orally administered to the patient may result in minimal plasma concentration of the oligonucleotide in the patient. In another embodiment, disclosed formulations, when orally administered to a patient, topically deliver to the colon or rectum of a patient, e.g. to an affected or diseased site of a patient.

### Parenteral Administration

The pharmaceutical compositions of the present disclosure may be formulated for parenteral administration, e.g., formulated for injection via the intravenous, intramuscular, subcutaneous, intralesional, or intraperitoneal routes. The preparation of an aqueous composition, such as an aqueous pharmaceutical composition containing a SMAD7 antisense oligonucleotide (e.g., an antisense oligonucleotide of SEQ ID NO: 1, 2, 3, 4, 5, or 6, or a pharmaceutically acceptable salt thereof), will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

In certain embodiments, the formulation of a SMAD7 antisense oligonucleotide (e.g., an antisense oligonucleotide of SEQ ID NO: 1, 2, 3, 4, 5, or 6, or a pharmaceutically acceptable salt thereof) is for use as a parenteral pharmaceutical composition. In certain embodiments, the formulation of a SMAD7 antisense oligonucleotide (e.g., an antisense oligonucleotide of SEQ ID NO: 1 or 2, or a pharmaceutically acceptable salt thereof) is for use as a parenteral pharmaceutical composition. In certain embodiments, the formulation of a SMAD7 antisense oligonucleotide (e.g., an antisense oligonucleotide of SEQ ID NO: 1, 2, 3, 4, 5, or 6, or a pharmaceutically acceptable salt thereof) is suitable for subcutaneous administration. In certain embodiments, the pharmaceutical composition/formulation for parenteral or subcutaneous administration includes a SMAD7 antisense oligonucleotide (e.g., an antisense oligonucleotide of SEQ ID NO: 1 or 2, or a pharmaceutically acceptable salt thereof). In certain embodiments, the pharmaceutical composition/formulation for parenteral or subcutaneous administration includes a SMAD7 antisense oligonucleotide (e.g., an antisense oligonucleotide of SEQ ID NO: 5 or 6, or a pharmaceutically acceptable salt thereof).

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds or pharmacologically acceptable salts thereof can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose any bland fixed oil can be employed including synthetic mono- or diglycerides. In addition, fatty acids such as oleic acid can be used in the preparation of injectables. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In embodiments, the SMAD7 inhibitor may be suspended in a carrier fluid including 1% (w/v) sodium carboxymethylcellulose and 0.1% (v/v) TWEEN™ 80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the disclosure may be prepared by incorporating an amount of a SMAD7 antisense oligonucleotide (e.g., an antisense oligonucleotide of SEQ ID NO: 1, 2, 3, 4, 5, or 6, or a pharmaceutically acceptable salt thereof), e.g., about 0.5% to about 70%, e.g., about 0.5% to about 10%, or about 1% to about 20%, by weight of the SMAD7 antisense oligonucleotide, in the required amount of the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly, concentrated solutions for intramuscular injection is also contemplated. In this regard, the use of DMSO as solvent is preferred as this will result in extremely rapid penetration, delivering high concentrations of the SMAD7 antisense oligonucleotide (e.g., an antisense oligonucleotide of SEQ ID NO: 1, 2, 3, 4, 5, or 6, or a pharmaceutically acceptable salt thereof) e.g., about 0.5% to about 70%, about 0.5% to about 10%, or about 1% to about 20%, by weight of an antisense oligonucleotide, to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium 10 carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose and the like.

In an exemplary embodiment, a pharmaceutical composition for subcutaneous administration of a SMAD7 antisense oligonucleotide includes an antisense oligonucleotide such as that represented by SEQ ID NO: 6, or a pharmaceutically acceptable salt thereof (such as a sodium salt), and a pharmaceutically acceptable carrier.

### Topical Formulation and Use

The present disclosure provides topical formulations of a SMAD7 antisense oligonucleotide (e.g., an antisense oligonucleotide of SEQ ID NO: 1, 2, 3, 4, 5, or 6) or a pharmaceutically acceptable salt thereof, including about 1% (w/w) to about 99% (w/w) of the oligonucleotide or a pharmaceutically acceptable salt thereof, and a pharmaceutically acceptable carrier. In embodiments, the present disclosure provides a topical formulation of a SMAD7 antisense oligonucleotide (e.g., an antisense oligonucleotide of SEQ ID NO: 1 or 2) or a pharmaceutically acceptable salt thereof, including about 1% (w/w) to about 99% (w/w) of the oligonucleotide or a pharmaceutically acceptable salt thereof, and a pharmaceutically acceptable carrier. In embodiments, the present disclosure provides a topical formulation of a SMAD7 antisense oligonucleotide (e.g., an antisense oligonucleotide of SEQ ID NO: 5 or 6) or a pharmaceutically acceptable salt thereof, including about 1% (w/w) to about 99% (w/w) of the antisense oligonucleotide or a pharmaceutically acceptable salt thereof, and a pharmaceutically acceptable carrier.

In embodiments, the formulation of a SMAD7 antisense oligonucleotide (e.g., SEQ ID NO: 1, 2, 3, 4, 5, or 6, or a pharmaceutically acceptable salt thereof) may be formulated as a gel, a cream, an ointment, a liquid, or a patch dosage form. The formulation of a SMAD7 antisense oligonucleotide (e.g., an antisense oligonucleotide of SEQ ID NO: 1, 2, 3, 4, 5, or 6, or a pharmaceutically acceptable salt thereof) may be formulated such that upon applying to a skin of a subject the formulation forms a patch.

For example, the topical formulations include or may include about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, about 90% to about 95%, or about 95% to about 99% of a SMAD7 antisense oligonucleotide (e.g., an antisense oligonucleotide of SEQ ID NO: 1, 2, 3, 4, 5, or 6) or a pharmaceutically acceptable salt thereof.

The topical formulations of the present disclosure may be formulated as a liquid, a solution, an emulsion, a cream, a lotion, a suspension, a triturate, a gel, a jelly, a foam, a paste, an ointment, a shampoo, an adhesive, a patch, or the like. In certain embodiments, the topical formulations of the present disclosure may be formulated as a liquid. In certain embodiments, the topical formulations of the present disclosure may be formulated as a solution. In certain embodiments, the topical formulations of the present disclosure may be formulated as an emulsion. In certain embodiments, the topical formulations of the present disclosure may be formulated as a cream. In certain embodiments, the topical formulations of the present disclosure may be formulated as a lotion. In certain embodiments, the topical formulations of the present disclosure may be formulated as a suspension. In certain embodiments, the topical formulations of the present disclosure may be formulated as a triturate. In certain embodiments, the topical formulations of the present disclosure may be formulated as a gel. In certain embodiments, the topical formulations of the present disclosure may be formulated as a jelly. In certain embodiments, the topical formulations of the present disclosure may be formulated as a foam. In certain embodiments, the topical formulations of the present disclosure may be formulated as a paste. In certain embodiments, the topical formulations of the present disclosure may be formulated as an ointment. In certain embodiments, the topical formulations of the present disclosure may be formulated as a shampoo. In certain embodiments, the topical formulations of the present disclosure may be formulated as an adhesive. In certain embodiments, the topical formulations of the present disclosure may be formulated as a patch. In certain embodiments, the topical formulations of the present disclosure upon application to a skin of a subject may form a patch.

The topical formulations (e.g., liquid, solution, emulsion, cream, lotion, suspension, triturate, gel, jelly, foam, past, ointment, shampoo, adhesive, patch, and the like) of a SMAD7 antisense oligonucleotide (e.g., an antisense oligonucleotide of SEQ ID NO: 1, 2, 3, 4, 5, or 6, or a pharmaceutically acceptable salt thereof) are or may be suitable for treating, preventing, and/or ameliorating a skin inflammation. In certain embodiments, the skin inflammation is psoriatic-like lesions or psoriasis. In certain embodiments, the skin inflammation is a pediatric skin inflammation.

Daily topical administration of the SMAD7 antisense oligonucleotide (e.g., an antisense oligonucleotide of SEQ ID NO: 1, 2, 3, 4, 5, or 6) or a pharmaceutically acceptable salt thereof, formulated as a topical formulation (e.g., a liquid, a solution, an emulsion, a cream, a lotion, a suspension, a triturate, a gel, a jelly, a foam, a paste, an ointment, a shampoo, an adhesive, a patch, and the like), once per day in the morning, and/or once per day in the evening during a treatment period which may be between one week, two weeks, one month, two months, or three months and one year may be effective to reduce the psoriatic lesions. The topical formulation (e.g., liquid, solution, emulsion, cream, lotion, suspension, triturate, gel, jelly, foam, past, ointment, shampoo, adhesive, patch, and the like) may be administered in an amount of between about 1.0 ml/5 cm² and 1.0 ml/50 cm², or between about 1.0 ml/5 cm² and 50 ml/50 cm², or between about 1.0 ml/5 cm² and 100 ml/50 cm².

In embodiments, the topical formulation (e.g., liquid, solution, emulsion, cream, lotion, suspension, triturate, gel, jelly, foam, past, ointment, shampoo, adhesive, patch, and the like) may be a mixture of both the SMAD7 antisense oligonucleotide (e.g., an antisense oligonucleotide of SEQ ID NO: 1, 2, 3, 4, 5, or 6) or a pharmaceutically acceptable salt thereof and a second therapeutic agent.

The topical formulation (e.g., liquid, solution, emulsion, cream, lotion, suspension, triturate, gel, jelly, foam, past, ointment, shampoo, adhesive, patch, and the like) of SMAD7 antisense oligonucleotide (e.g., an antisense oligonucleotide of SEQ ID NO: 1, 2, 3, 4, 5, or 6) or a pharmaceutically acceptable salt thereof, may also include one or more cosmetically or pharmaceutically acceptable carriers/excipients. Suitable carriers/excipients that may be used in the topical formulations discussed herein are known in the art and include, but are not limited to, solubilizers such as C₂ to C₈ straight and branched chain alcohols, diols and triols, moisturizers and humectants such as glycerine, amino acids and amino acid derivatives, polyaminoacids and derivatives, pyrrolidone carboxylic acids and its salts and derivatives, surfactants such as sodium laureth sulfate, sorbitan monolaurate, emulsifiers such as cetyl alcohol, stearyl alcohol, thickeners such as methyl cellulose, ethyl cellulose, hydroxymethylcellulose, hydroxypropylcellulose, polyvinylpyrollidone, polyvinyl alcohol and acrylic polymers.

The topical formulation (e.g., liquid, solution, emulsion, cream, lotion, suspension, triturate, gel, jelly, foam, past, ointment, shampoo, adhesive, patch, and the like) of a SMAD7 antisense oligonucleotide (e.g., an antisense oligonucleotide of SEQ ID NO: 1, 2, 3, 4, 5, or 6) or a pharmaceutically acceptable salt thereof, may also include propylene glycol. The propylene glycol may be present in the formulation between about 1% w/w to about 25% w/w. Additionally the topical formulation (e.g., liquids, solutions, emulsions, creams, lotions, suspensions, triturates, gels, jellies, foams, pastes, ointments, shampoos, adhesives, patches, and the like) of SMAD7 antisense oligonucleotide (e.g., an antisense oligonucleotide of SEQ ID NO: 1, 2, 3, 4, 5, or 6) or a pharmaceutically acceptable salt thereof, may also include ethanol and/or polyethylene glycol 300. The ethanol may be present in the formulation between about 1% w/w to about 25% w/w. The polyethylene glycol 300 may be present in the range of between about 1% w/w to about 80% w/w. In addition the topical formulation may include at least one moisturizer/humectant.

The topical formulation (e.g., liquid, solution, emulsion, cream, lotion, suspension, triturate, gel, jelly, foam, past, ointment, shampoo, adhesive, patch, and the like) of SMAD7 antisense oligonucleotide (e.g., SEQ ID NO: 1, 2, 3, 4, 5, or 6) or a pharmaceutically acceptable salt thereof, may be applied to the skin by any means known in the art including, but not limited to, by an aerosol, spray, pump-pack, brush, swab, or other applicator. In embodiments, the applicator provides either a fixed or variable metered dose application such as a metered dose aerosol, a stored-energy metered dose pump or a manual metered dose pump. In embodiments, the drug delivery system is applied to the skin of the human or animal covering a delivery surface area between about 10 and 800 cm², more preferably between about 10 and 400 cm², and most preferably about 10 and 200 cm². The application may be performed by means of a topical metered dose spray combined with an actuator nozzle shroud which together accurately control the amount and/or uniformity of the dose applied. One function of the shroud may be to keep the nozzle at a pre-determined height above, and perpendicular to, the skin to which the drug delivery system is being applied. This function may also be achieved by means of a spacer-bar or the like. Another function of the shroud is to enclose the area above the skin in order to prevent or limit bounce-back and/or loss of the drug delivery system to the surrounding environment. In embodiments, the area of application defined by the shroud is substantially circular in shape.

In certain embodiments, the drug delivery system may be a unit volume dispenser with or without a roll-on or other type of applicator. It may also be necessary to apply a number of dosages on untreated skin to obtain the desired result.

Topical formulations (e.g., liquid, solution, emulsion, cream, lotion, suspension, triturate, gel, jelly, foam, past, ointment, shampoo, adhesive, patch, and the like) of SMAD7 antisense oligonucleotide (e.g., an antisense oligonucleotide of SEQ ID NO: 1, 2, 3, 4, 5, or 6) or a pharmaceutically acceptable salt thereof, of the present disclosure may contain a pharmaceutically acceptable topical carrier and a pharmacologically active base, without any additional pharmacologically active agents. The formulation may be may be prepared so as to contain liposomes, micelles, and/or microspheres. In certain embodiments, a topical formulation may be aqueous, i.e., contain water, or may be nonaqueous and optionally used in combination with an occlusive overlayer so that moisture evaporating from the body surface is maintained within the formulation upon application to the body surface and thereafter.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. In embodiments, water-soluble ointment bases are prepared from polyethylene glycols of varying molecular weight; again, see Remington: The Science and Practice of Pharmacy for further information.

Creams, as also well known in the art, are viscous liquids or semisolid emulsions, either oil-in-water or water-in-oil. Cream bases are water-washable, and contain an oil phase, an emulsifier, and an aqueous phase. The oil phase, also called the “internal” phase, is generally included of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

Gels are semisolid, suspension-type systems. Single-phase gels contain organic macromolecules distributed substantially uniformly throughout the carrier liquid, which is typically aqueous, but also contains an alcohol and, optionally, an oil. In embodiments, “organic macromolecules,” i.e., gelling agents, are crosslinked acrylic acid polymers such as the “carbomer” family of polymers, e.g., carboxypolyalkylenes that may be obtained commercially under the CARBOPOL™ trademark. Hydrophilic polymers such as polyethylene oxides, polyoxyethylene-polyoxypropylene copolymers, and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin may also be included. In order to prepare a uniform gel, dispersing agents such as alcohol or glycerin can be added, or the gelling agent can be dispersed by trituration, mechanical mixing, or stirring, or combinations thereof.

Lotions are preparations to be applied to the skin surface without friction, and are typically liquid or semiliquid preparations in which solid particles, including the active agent, are present in a water or alcohol base. Lotions are usually suspensions of solids, and preferably, for the present purpose, include a liquid oily emulsion of the oil-in-water type. In embodiments, lotions are used for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like.

Pastes are semisolid dosage forms in which the SMAD7 antisense oligonucleotide (e.g., an antisense oligonucleotide of SEQ ID NO: 1, 2, 3, 4, 5, or 6) or a pharmaceutically acceptable salt thereof is suspended in a suitable base. Depending on the nature of the base, pastes are divided between fatty pastes or those made from a single-phase aqueous gels. The base in a fatty paste is generally petrolatum, hydrophilic petrolatum, or the like. The pastes made from single-phase aqueous gels generally incorporate carboxymethylcellulose or the like as a base.

Formulations may also be prepared with liposomes, micelles, and microspheres. Liposomes are microscopic vesicles having a lipid wall including a lipid bilayer, and can be used as drug delivery systems herein as well. Generally, liposome formulations are poorly soluble or insoluble pharmaceutical agents. Liposomal preparations for use in the instant disclosure may include cationic (positively charged), anionic (negatively charged), and neutral preparations. Cationic liposomes are readily available. For example, N[1-2,3-dioleyloxy)propyl]-N,N,N-triethylammonium (DOTMA) liposomes are available under the trade name LIPOFECTIN™. (ThermoFisher). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE), among others. These materials can also be mixed with DOTMA in appropriate ratios. Methods for making liposomes using these materials are well known in the art.

Micelles are known in the art as included of surfactant molecules arranged so that their polar headgroups form an outer spherical shell, while their hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10, and nonoxynol 30. Micelle formulations can be used in conjunction with the present disclosure either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a drug or drug-containing formulation. Microspheres are generally, although not necessarily, formed from synthetic or naturally occurring biocompatible polymers, but may also be included of charged lipids such as phospholipids. Preparation of microspheres is well known in the art and described in the pertinent texts and literature.

Various additives, known to those skilled in the art, may be included in the topical formulations. For example, solvents, including relatively small amounts of alcohol, may be used to solubilize certain formulation components. In embodiment, the formulation includes a suitable enhancer, e.g., but are not limited to, ethers such as diethylene glycol monoethyl ether (available commercially as TRANSCUTOL™) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin (U.S. Pat. No. 4,783,450); alcohols such as ethanol, propanol, octanol, benzyl alcohol, and the like; polyethylene glycol and esters thereof such as polyethylene glycol monolaurate (PEGML); amides and other nitrogenous compounds such as urea, dimethylacetamide (DMA), dimethylformamide (DMF), 2-pyrrolidone, 1-methyl-2-pyrrolidone, ethanolamine, diethanolamine, and triethanolamine; terpenes; alkanones; and organic acids, particularly citric acid and succinic acid. AZONE™ and sulfoxides such as DMSO and C₁₀ MSO may also be used.

The present formulations may also include conventional additives such as opacifiers, antioxidants, fragrance, colorant, gelling agents, thickening agents, stabilizers, surfactants, and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

The formulations may also contain irritation-mitigating additives to minimize or eliminate the possibility of skin irritation or skin damage resulting from the pharmacologically active base or other components of the composition. Suitable irritation-mitigating additives include, for example: a-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present formulations at a concentration effective to mitigate irritation or skin damage, typically representing not more than about 20 wt. %, more typically not more than about 5 wt. %, of the composition.

The SMAD7 antisense oligonucleotide (e.g., an antisense oligonucleotide of SEQ ID NO: 1, 2, 3, 4, 5, or 6) or a pharmaceutically acceptable salt thereof may also be administered through the skin or mucosal tissue using a conventional skin patch, in which the SMAD7 antisense oligonucleotide is contained within a laminated structure that serves as a drug delivery device to be affixed to the body surface. In such a structure, the topical formulation is contained in a layer, or “reservoir,” underlying an upper backing layer. The laminated structure may contain a single reservoir, or it may contain multiple reservoirs.

In certain embodiments, the reservoir may include a polymeric matrix of a pharmaceutically acceptable adhesive material that serves to affix the system to the skin during delivery of SMAD7 antisense oligonucleotide (e.g., an antisense oligonucleotide of SEQ ID NO: 1, 2, 3, 4, 5, or 6) or a pharmaceutically acceptable salt thereof. In embodiments, the adhesive material may be a pressure-sensitive adhesive (PSA) that is suitable for long-term skin contact, and that is physically and chemically compatible with the SMAD7 antisense oligonucleotide (e.g., an antisense oligonucleotide of SEQ ID NO: 1, 2, 3, 4, 5, or 6) or a pharmaceutically acceptable salt thereof, and any carriers, vehicles, or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). Preferred adhesives are polyisobutylenes.

The backing layer functions as the primary structural element of the transdermal system and provides the device with flexibility and, preferably, occlusivity. The material used for the backing layer should be inert and incapable of absorbing drug, base, or other components of the formulation contained within the device. The backing preferably includes a flexible elastomeric material that serves as a protective covering to prevent loss of drug and/or vehicle via transmission through the upper surface of the patch, and preferably imparts a degree of occlusivity to the system, such that the area of the body surface covered by the patch becomes hydrated during use. The material used for the backing layer should permit the device to follow the contours of the skin and be worn comfortably on areas of skin such as at joints or other points of flexure that are normally subjected to mechanical strain, with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

The method of delivery of a topical formulation of a SMAD7 antisense oligonucleotide (e.g., an antisense oligonucleotide of SEQ ID NO: 1, 2, 3, 4, 5, or 6) or a pharmaceutically acceptable salt thereof may vary, but may involve application of a formulation of the disclosure to an area of body surface affected with or at risk of being affected with an inflammation and/or psoriasis. A cream, ointment, or lotion may be spread on the affected surface and gently rubbed in. A solution may be applied in the same way, but more typically will be applied with a dropper, swab, or the like, and carefully applied to the affected areas.

The dose regimen of a topical formulation of a SMAD7 antisense oligonucleotide (e.g., an antisense oligonucleotide of SEQ ID NO: 1, 2, 3, 4, 5, or 6) or a pharmaceutically acceptable salt thereof, will depend on a number of factors that may readily be determined, such as severity of the dermatosis and responsiveness of the condition to be treated, but will normally be one or more doses per day, with a course of treatment lasting from several days to several months, or until a cure is effected or a diminution of disease state is achieved. One of ordinary skill may readily determine optimum dosages, dosing methodologies, and repetition rates. In general, it is contemplated that the formulation will be applied one to four times daily. With a skin patch, the device is generally maintained in place on the body surface throughout a drug delivery period, typically in the range of 8 to 72 hours, and replaced as necessary.

It is to be understood that while the disclosure has been described in conjunction with the preferred specific embodiments thereof, the foregoing description is intended to illustrate and not limit the scope of the disclosure. Other aspects, advantages, and modifications will be apparent to those skilled in the art to which the disclosure pertains. Furthermore, the practice of the present disclosure will employ, unless otherwise indicated, conventional techniques of drug formulation, particularly topical and transdermal drug formulation, which are within the skill of the art. Such techniques are fully explained in the literature. See Remington: The Science and Practice of Pharmacy, cited supra, as well as Goodman & Gilman's The Pharmacological Basis of Therapeutics, 9th Ed. (New York: McGraw-Hill, 1996).

### Dosage Regimen

The formulation of a SMAD7 antisense oligonucleotide (e.g., an antisense oligonucleotide of SEQ ID NO: 1, 2, 3, 4, 5, or 6) or a pharmaceutically acceptable salt thereof, provided in the present disclosure may be administered or is suitable for administration before and/or after symptoms of moderate to severe skin inflammation, a psoriasis-like lesion, and/or psoriasis is developed.

The formulation of the present disclosure (including e.g., SEQ ID NO: 1, 2, 3, 4, 5, or 6 or a pharmaceutically acceptable salt thereof) may be administered or is suitable for administration about every 6 hours, about every 12 hours, about every 24 hours, about every 48 hours, about every 72 hours, every day, two-times a week, once in 2 weeks, or once a month.

Dosing frequency can vary, depending on factors such as route of administration, dosage amount and the disease being treated. Exemplary dosing frequencies are once per day, once per week and once every two weeks. In certain embodiments, dosing is once per day for 7 days.

In certain embodiments, formulations include dosage forms that include or consist essentially of about 35 mg to about 500 mg of a SMAD7 antisense oligonucleotide (including e.g., an antisense oligonucleotide of SEQ ID NO: 1, 2, 3, 4, 5, or 6) or a pharmaceutically acceptable salt thereof). For example, formulations that include about 35 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, or about 250 mg of a SMAD7 antisense oligonucleotide are contemplated herein. In certain embodiments, a formulation may include about 40 mg, about 80 mg, or about 160 mg of a SMAD7 antisense oligonucleotide. In certain embodiments, a formulation may include at least about 100 μg of a SMAD7 antisense oligonucleotide. For example, formulations may include about 0.1 mg, about 0.2 mg, about 0.3 mg, about 0.4 mg, about 0.5 mg, about 1 mg, about 5 mg, about 10 mg, about 15 mg, about 20 mg, or about 25 mg of a SMAD7 antisense oligonucleotide.

The amount administered will depend on variables such as the type and extent of disease or indication to be treated, the overall health and size of the patient, the in vivo potency of the antisense oligonucleotide, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study designed to run from 40 mg to 160 mg.

In embodiments, a patient having a skin inflammation, e.g., psoriasis, will be administered an initial dose of an SMAD7 antisense oligonucleotide, for instance, a SMAD7 antisense oligonucleotide. As used herein, “initial dose” refers to a dose of an SMAD7 antisense oligonucleotide administered to a patient having a skin inflammation, e.g., psoriasis, in a series of doses. A series of doses may include one or more doses. For instance, a series of doses may include a single dose of an SMAD7 antisense oligonucleotide or more than a single dose of an SMAD7 antisense oligonucleotide. An initial dose may be a dose of an SMAD7 antisense oligonucleotide administered to a patient prior to any later dose administered to the patient. For instance, an initial dose may be, but is not limited to, the first dose of an SMAD7 antisense oligonucleotide administered to a treatment-naïve patient. An initial dose may also be a first dose in any treatment cycle of the SMAD7 antisense oligonucleotide. For example, an initial dose may be the first dose of a first treatment cycle, of a second treatment cycle, or of any subsequent treatment cycles. Alternatively, an “initial dose” may be the first dose administered to a patient after analyzing levels of p-SMAD3, α-SMA, or TGF-β and/or another biomarker or biomarkers in a patient, or may be the most recently administered dose before a determination of the levels of p-SMAD3, α-SMA, or TGF-β and/or another biomarker or biomarkers in a patient.

In embodiments of the disclosure, a patient having a skin inflammation, e.g., psoriasis, may be administered a subsequent dose of an SMAD7 antisense oligonucleotide, for instance, a SMAD7 antisense oligonucleotide. As used herein, “subsequent dose” refers to a dose of an SMAD7 antisense oligonucleotide administered to a patient having a skin inflammation, e.g., psoriasis, after administration of a prior dose, for example, an initial dose. Thus, a subsequent dose may be administered to a patient having a skin inflammation, e.g., psoriasis, in a series of doses including two or more doses. Furthermore, in some instances, the amount of a subsequent dose may be calibrated with respect to an initial dose or a prior dose, such that a subsequent dose is greater, equal to, or lesser than a prior dose. Calibration of the amount of a subsequent dose may be based on levels or changes in levels of p-SMAD3, α-SMA, or TGF-β and/or another biomarker or biomarkers in a patient having a skin inflammation, e.g., psoriasis, for instance: levels of p-SMAD3, α-SMA, or TGF-β in a patient having a skin inflammation, e.g., psoriasis, analyzed prior to or after a prior dose, for instance, an initial dose; or changes in p-SMAD3, α-SMA, or TGF-β levels in a patient having a skin inflammation, e.g., psoriasis, before and after a prior dose, for instance, an initial dose. A subsequent dose may be a dose administered to a patient having a skin inflammation, e.g., psoriasis, after a first dose, for instance, an initial dose, of an SMAD7 antisense oligonucleotide administered to a patient having a skin inflammation, e.g., psoriasis. A subsequent dose may also be a dose administered after a prior dose of an SMAD7 antisense oligonucleotide administered to a patient having a skin inflammation, e.g., psoriasis, for instance, a dose administered after a prior dose in the same round of treatment or a different round of treatment, for instance, a previous round of treatment. A subsequent dose may be a subsequent dose with respect to any prior dose, for instance, a prior dose immediately preceding the subsequent dose or a prior dose followed by one or more doses administered prior to administration of the subsequent dose.

Patients treated using an above method may or may not have detectable skin inflammation, e.g., psoriasis. In embodiments, the patient has at least about a 5%, about a 10%, about a 20%, about a 30%, about a 40% or even about a 50% or more reduction in the amount of skin inflammation, e.g., psoriasis, present in the patient after administering a specific inhibitor of SMAD7 (e.g., a SMAD7 antisense oligonucleotide including e.g., an antisense oligonucleotide of SEQ ID NO: 1, 2, 3, 4, 5, or 6 or a pharmaceutically acceptable salt thereof), after e.g., 1 day, 2 days, 1 week, 1 month, or 6 months, or more. Administering an inhibitor of SMAD7 may be on, e.g., at least a daily basis. The delay of clinical manifestation of skin inflammation, e.g., psoriasis, in a patient as a consequence of administering an inhibitor of SMAD7 may be at least e.g., 6 months, 1 year, 18 months or even 2 years or more as compared to a patient who is not administered an inhibitor of SMAD7.

### Combination Therapy

The formulation or method the present disclosure provides administration of a formulation of a SMAD7 antisense oligonucleotide (e.g., an antisense oligonucleotide of SEQ ID NO: 1, 2, 3, 4, 5, or 6) or a pharmaceutically acceptable salt thereof, to a subject who is refractory to a first therapy.

The first therapy may be but not limited to cyclosporine, corticosteroid, and/or a fumaric acid ester (e.g., dimethyl fumarate) or derivatives thereof.

In embodiments, a subject who is refractory to the first treatment is treated with a formulation of a SMAD7 antisense oligonucleotide (e.g., an antisense oligonucleotide of SEQ ID NO: 1, 2, 3, 4, 5, or 6) or a pharmaceutically acceptable salt thereof, concurrently or subsequent to the first therapy.

In embodiments, methods provided herein may further include administering at least one other agent that is directed to treatment of diseases and disorders disclosed herein (e.g., psoriasis). In certain embodiments, contemplated other agents may be co-administered (e.g., sequentially or simultaneously).

Agents contemplated include immunosuppressive agents including glucocorticoids, cytostatics, antibodies, agents acting on immunophilins, interferons, opioids, TNF binding proteins, mycophenolate, and small biological agents. For example, contemplated immunosuppressive agents include, but are not limited to: tacrolimus, cyclosporine, pimecrolimus, sirolimus, everolimus, mycophenolic acid, fingolimod, dexamethasone, fludarabine, cyclophosphamide, methotrexate, azathioprine, leflunomide, teriflunomide, anakinra, anti-thymocyte globulin, anti-lymphocyte globulin, muromonab-CD3, afutuzumab, rituximab, teplizumab, efalizumab, daclizumab, basiliximab, adalimumab, infliximab, and etanercept.

### EXAMPLES

The disclosure is further illustrated by the following examples. The examples are provided for illustrative purposes only, and are not to be construed as limiting the scope or content of the disclosure in any way.

### Example 1: Up-Regulation of SMAD7 in Psoriatic Skins

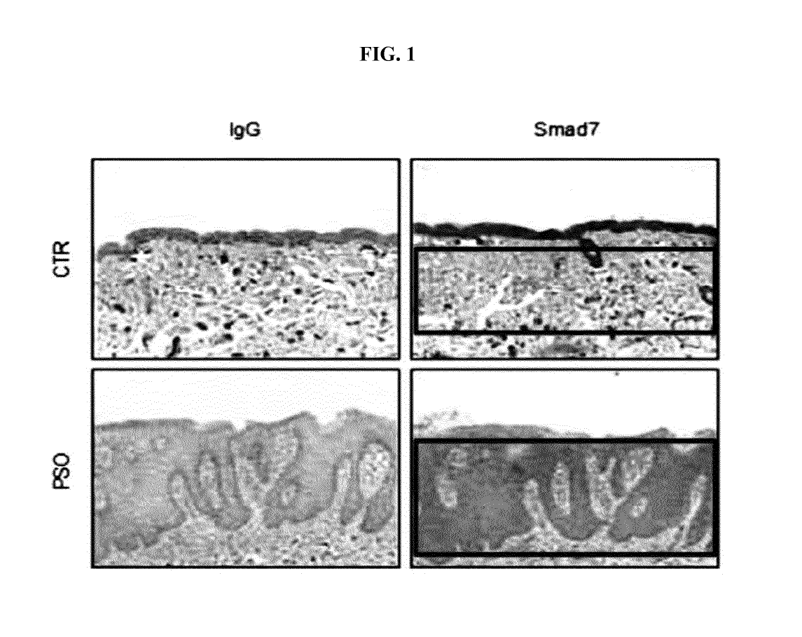

Expression of SMAD7 in human psoriatic lesions was tested with immune-staining skin specimens taken from psoriatic area of patients with psoriasis (PSO) and healthy area of control subjects (CTR). FIG. 1 is representative of four separate experiments in which sections of 7 PSO patients and 5 controls were analysed. Staining with isotype control IgG is also shown. The results show that the SMAD7 expression was upregulated in psoriatic tissues.

### Example 2: SMAD7 Sustains Keratinocyte Proliferation

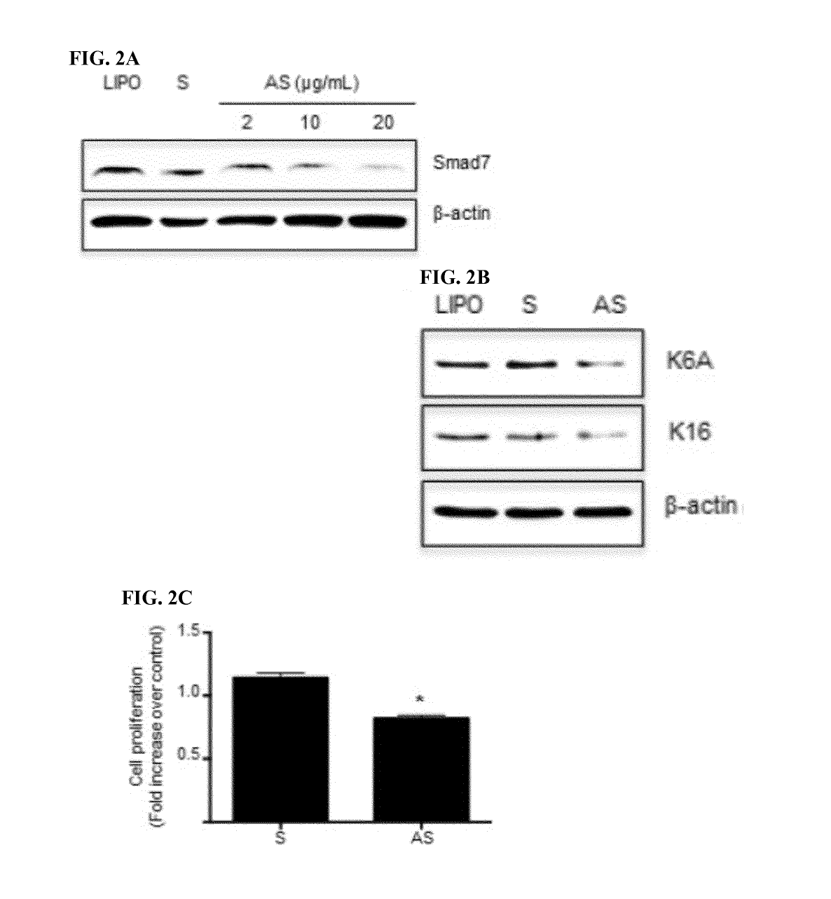

SMAD7 was shown to sustain keratinocyte proliferation. FIG. 2A shows an experiment in which dose-dependent reduction of SMAD7 protein expression in HaCaT cells treated with increasing doses of SMAD7 antisense (AS) oligonucleotide was observed. HaCaT cells were transfected with lipofectamine (LIPO) in the presence or absence of SMAD7 sense oligonucleotide (S) (20 μg/ml) or increasing doses (2-20 μg/ml) of SMAD7 AS for 24h and SMAD7 protein was evaluated by Western blotting. β-actin was used as loading control. One of three representative experiments, in which similar results were obtained, is shown in FIG. 2A. FIG. 2B shows results of an experiment in which HaCaT cells were treated with SMAD7 antisense oligonucleotide (AS) (20 μg/ml) and resulted in a reduction of keratin (K) 6A and K16 protein expression. Cells were transfected with lipofectamine (LIPO) in the presence or absence of SMAD7 sense oligonucleotide (S) or AS (20 μg/ml) for 24 h and K6A and K16 were evaluated by Western blotting. β-actin was used as loading control. One of three representative experiments, in which similar results were obtained, is shown. FIG. 2C represents experiments in which knockdown of SMAD7 was evaluated for its effect on cell proliferation. Knockdown of SMAD7 was observed to reduce HaCaT proliferation. Cells were transfected with lipofectamine (LIPO) in the presence or absence of SMAD7 S or AS (20 μg/ml) for 24 h and 5-bromo-2-deoxyuridine (BrdU) was added to the cell cultures 6 hours before the end of the treatment. BrdU-positive cells were evaluated by ELISA using a commercial colorimetric assay. Data are expressed as fold-increase over control (LIPO) and indicate mean±SD of 3 separate experiments. (SMAD7 S-transfected cells versus SMAD7 AS-transfected cells, *P<0.01). These results demonstrate that SMAD7 knockdown resulted in decreased keratin protein levels and decreased cell proliferation in HaCaT cells.

### Example 3: SMAD7 Inhibition Arrests S-Phase of the Cell Cycle

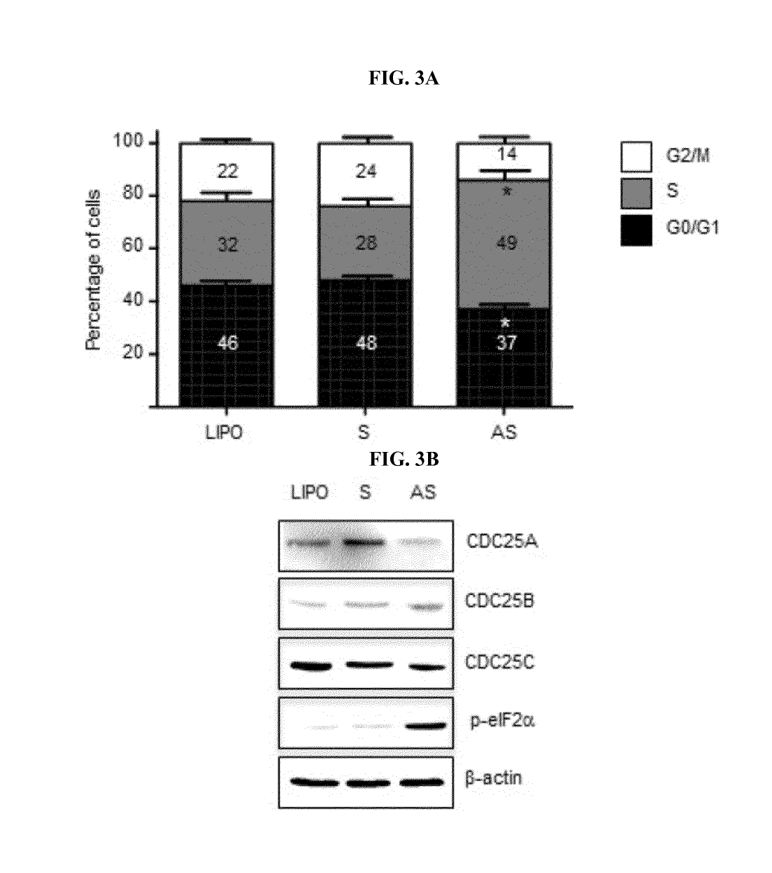

Effects of SMAD7 knockdown in cell cycle were tested in HaCaT cells. SMAD7 knockdown was observed to induce HaCaT cells to arrest in S-phase of the cell cycle. FIG. 3A shows effects on cells that were transfected with lipofectamines(LIPO) in the presence or absence of SMAD7 S or AS (20 μg/ml) for 24 h after which cell cycle distribution was assessed by flow cytometry. Values indicate the percentages of cells in the different phases of cell cycle and indicate mean±SD of 3 separate experiments. A significant increase in the number of cells that accumulate in S phase and a significant decrease in the number of cells in G0/G1 is seen in SMAD7 AS-transfected cells as compared with SMAD7 S-transfected cells (*P<0.01). FIG. 3B shows Western blot results following SMAD7 knockdown in HaCaT cells. SMAD7 knockdown in HaCaT cells enhanced eIF2α phosphorylation and down-regulated CDC25A expression. Cells were transfected with lipofectamine in the presence or absence of SMAD7 S or AS (20 μg/ml) for 24 h and CDC25A, CDC25B and CDC25C expression was assessed by Western blotting. One of 3 representative experiments in which similar results were obtained is shown. These results demonstrate that SMAD7 knockdown resulted in S phase cell cycle arrest and modulation of proteins associated with cell cycle regulation.

### Example 4: SMAD7 Overexpression Results in Increased Expression of Keratin (K) 6A and 16

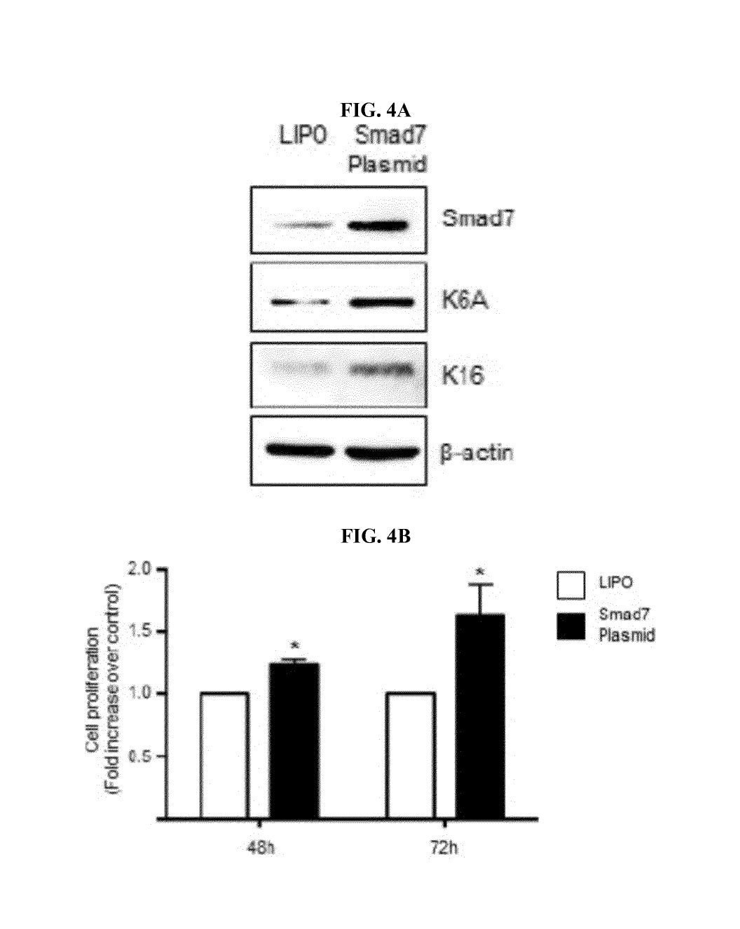

Effect of SMAD7 overexpression on the expression level of keratin (K) 6A and 16 was tested. The results show that SMAD7 overexpression increased keratin (K) 6A and 16 expression and positively regulated HaCaT proliferation. HaCaT cells were either incubated with lipofectamine (LIPO) or transfected with plasmid (P) DNA (1 μg/ml) containing SMAD7 gene sequence for 48 h (FIG. 4A). K6A and K16 expression was evaluated by Western blotting. β-actin was used as loading control (FIG. 4A). One of 3 separate experiments, in which similar results were obtained, is shown. Additionally, HaCaT cells were either incubated with LIPO or transfected with plasmid (P) DNA (1 μg/ml) containing SMAD7 gene sequence for 48 and 72 hours and 5-bromo-2-deoxyuridine (BrdU) was added to the cell cultures 6 hours before the end of the treatment (FIG. 4B). BrdU-positive cells were evaluated by ELISA using a commercial colorimetric assay. Data are expressed as fold-increase over control and indicate mean±SD of 3 separate experiments (*P<0.01). These results demonstrate that SMAD7 overexpression resulted in increased keratin protein expression and HaCaT cell proliferation.

### Example 5: SMAD7 Down-Regulation is Effective in Alleviating Symptoms of Psoriatic Symptoms

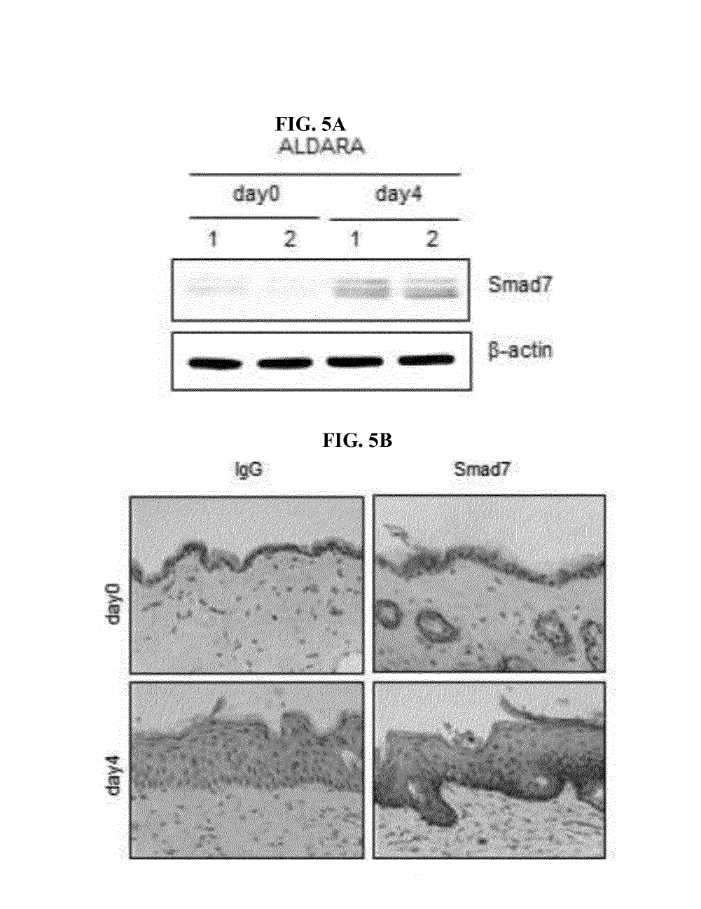

Topical treatment of skin with Aldara, a cream preparation containing 5% Imiquimod (IMQ) results in tumor regression in patients with non-melanoma skin cancer. IMQ is a ligand for the toll-like receptors TLR7 and TLR8. It is a potent immune activator that exacerbates psoriasis at both the local treated areas as well as distant sites which has led to the development of pre-clinical models of psoriasis using topically applied Aldara cream. In this example, the level of SMAD7 expression on Aldara-mediated psoriasis-like lesions was evaluated. SMAD7 expression increased in the skin of mice with Aldara-mediated psoriasis-like lesions (FIG. 5A). Aldara cream was applied daily on the shaved back skin of C57BL/6 mice. Mice were killed at day 4 and total proteins extracted were analysed for SMAD7 expression by Western blotting (FIG. 5A) and immunohistochemistry (FIG. 5B). The figures are representative of six separate experiments. β-actin was used as a loading control in Western blotting studies. Staining with isotype control IgG is also shown in panel FIG. 5B. The results confirm that in a psoriatic model system SMAD7 expression levels were increased following topical application of Aldara.

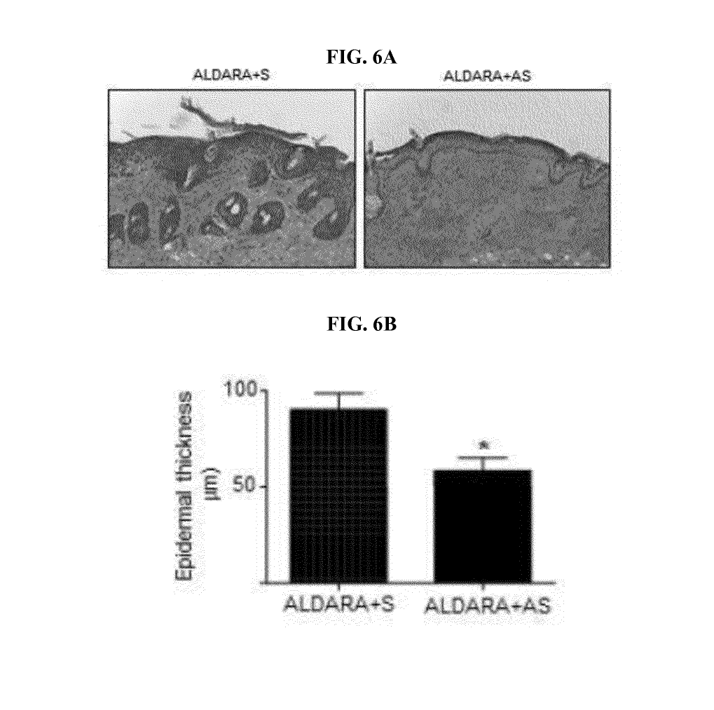

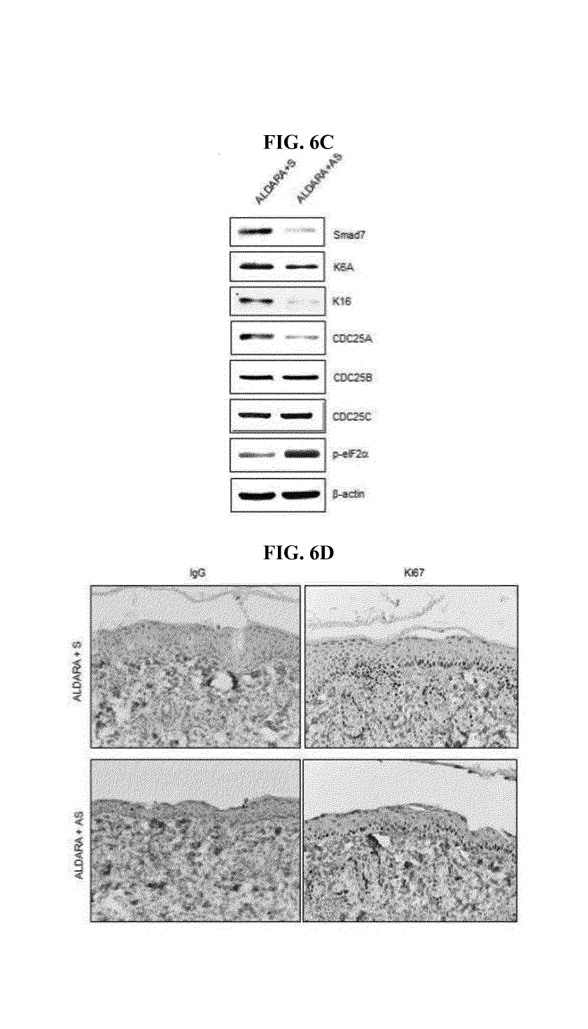

Effect of SMAD7 inhibition on skin thickness and keratinocyte proliferation was evaluated Inhibition of SMAD7 with a specific SMAD7 antisense oligonucleotide reduced skin thickness and keratinocyte proliferation (FIGS. 6A and 6B). Aldara cream was applied daily on the shaved back skin of C57BL/6 mice for 4 days and SMAD7 antisense (AS) or sense (S) (125 μg/mouse) oligonucleotides were topically applied each day starting 12 hours after Aldara treatment. Representative stainings with hematoxylin and eosin of skin sections of mice treated as above is shown in FIG. 6A. One of 3 separate experiments, in which 12 mice per group were analysed, is shown. Epidermal thickness was evaluated using skin sections of mice treated as above and data are expressed as mean±SD of all experiments (FIG. 6B). SMAD7 AS-treated mice vs SMAD7 S-treated mice *P<0.01. The results presented in FIG. 6A and FIG. 6B showed that administering SMAD7 antisense oligonucleotide reduced epidermal thickness associated with Aldara-associated psoriatic symptoms.

For the experiment shown in FIG. 6C, mice were treated as above and total proteins extracted from the skin were analysed for the indicated proteins by Western blotting. One representative experiment is shown. FIG. 6D shows representative immune-staining for Ki67 of skin sections of mice treated as indicated in FIG. 6A. Staining with isotype IgG is also shown. The results presented in FIG. 6C and FIG. 6D showed that administering SMAD7 antisense oligonucleotide reduced keratin protein (K6A and K16) expression levels and cell proliferation levels associated with Aldara-associated psoriatic symptoms.

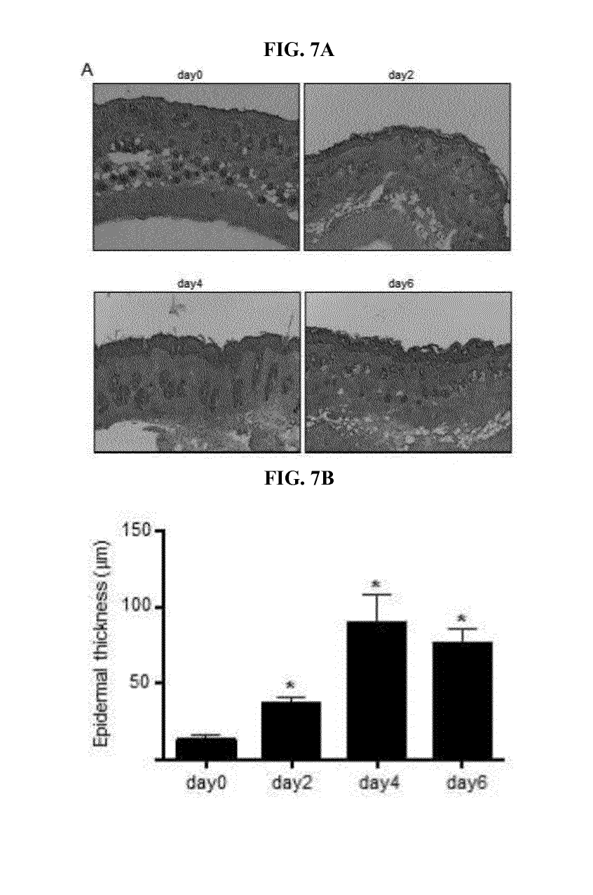

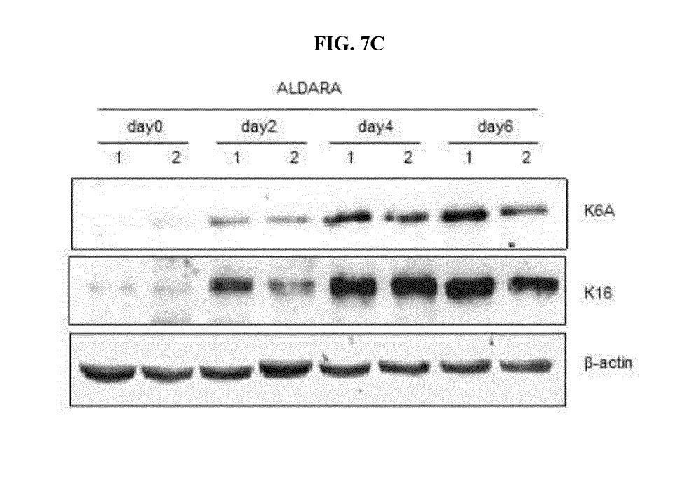

Epidermal thickness was evaluated in skin sections of mice treated with daily cutaneous administration of Aldara cream. Representative stainings with hematoxylin and eosin of skin sections of mice treated with Aldara cream for the indicated time points are shown in FIGS. 7A-7B. Data in FIG. 7C are expressed as mean±SD of 3 separate experiments. Control mice (day 0) vs Aldara-treated mice *P<0.05. FIG. 7C shows Keratin (K) 6A and K16 expression in total extracts of skin from mice treated as indicated in the experiment for FIG. 7A. One representative experiment is shown. β-actin was used as loading control. The results presented in FIGS. 7A-7C showed that in the Aldara model system for psoriasis or psoriatic symptoms, K16 and K6A are over-expressed following administration of Aldara, and supported the observation of FIGS. 5A-5B and FIGS. 6A-6D that SMAD7 antisense oligonucleotide administration resulted in reduced expression of K16 and K6A, thereby treatment, prevention, and/or amelioration of psoriasis or psoriatic symptoms.

### Example 6: Treatment, Prevention, and/or Ameliorating a Skin Inflammation (e.g., Psoriasis) or Symptoms

Subjects with symptoms of psoriasis or psoriatic-like lesions or subjects at risk of psoriasis or psoriatic-like lesions (e.g., subjects that were treated with another drug and are in partial or complete remission, and require secondary treatment in order to prevent re-appearance of the symptoms and/or the lesions) are administered with a SMAD7 antisense oligonucleotide. The subjects are administered an effective dose of SMAD7 antisense oligonucleotide to treat, prevent, and/or ameliorate psoriasis is by oral, topical, or parenteral mode of administration. The administration of SMAD7 antisense oligonucleotide results in treatment, prevention, and/or amelioration of psoriasis and/or a symptom thereof. The treating, preventing, and/or ameliorating psoriasis and/or a symptom thereof with SMAD7 antisense oligonucleotide is about 30% to 100% effective.

### INCORPORATION BY REFERENCE

The entire disclosure of each of the patent documents and scientific articles cited herein is incorporated by reference for all purposes.

### EQUIVALENTS

The disclosure can be embodied in other specific forms with departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

What is claimed is:

1. A method of treating, preventing, and/or ameliorating a skin inflammation or symptoms thereof in a subject in need thereof, comprising administering to the subject a pharmaceutical composition comprising an effective amount of a Mothers against decapentaplegic homolog 7 (SMAD7) antisense oligonucleotide or a pharmaceutically acceptable salt thereof, wherein upon administration the composition treats, prevents, and/or ameliorates the skin inflammation of the subject.

2. A method of treating, preventing, and/or ameliorating psoriasis or symptoms thereof in a subject in need thereof, comprising administering to the subject a pharmaceutical composition comprising an effective amount of a Mothers against decapentaplegic homolog 7 (SMAD7) antisense oligonucleotide or a pharmaceutically acceptable salt thereof, wherein upon administration the composition treats, prevents, and/or ameliorates the psoriasis or symptoms thereof of the subject.

3. A method of reducing epidermal hyperproliferation of keratinocytes in a subject in need thereof, comprising administering to the subject a pharmaceutical composition comprising an effective amount of a Mothers against decapentaplegic homolog 7 (SMAD7) antisense oligonucleotide or a pharmaceutically acceptable salt thereof, wherein upon administration the composition reduces epidermal hyperproliferation of keratinocytes of the subject.

4. A method of reducing skin thickness in a subject in need thereof, comprising administering to the subject a pharmaceutical composition comprising an effective amount of a Mothers against decapentaplegic homolog 7 (SMAD7) antisense oligonucleotide, wherein upon administration the composition reduces skin thickness of the subject.

5. A method of treating, preventing, and/or ameliorating psoriatic lesions and/or psoriasis-like skin inflammation in a subject in need thereof, comprising administering to the subject a pharmaceutical composition comprising an effective amount of a Mothers against decapentaplegic homolog 7 (SMAD7) antisense oligonucleotide or a pharmaceutically acceptable salt thereof, wherein upon administration the composition treats, prevents, and/or ameliorates the psoriatic lesions and/or psoriasis-like skin inflammation of the subject.

6. A method of maintaining remission of a skin inflammation and/or symptoms thereof, comprising administering to the subject a pharmaceutical composition comprising an effective amount of a Mothers against decapentaplegic homolog 7 (SMAD7) antisense oligonucleotide or a pharmaceutically acceptable salt thereof, wherein upon administration the composition maintains remission of the skin inflammation and/or symptoms thereof of the subject.

7. A method of slowing the progression of psoriatic arthritis and/or symptoms thereof, comprising administering to the subject a pharmaceutical composition comprising an effective amount of a Mothers against decapentaplegic homolog 7 (SMAD7) antisense oligonucleotide or a pharmaceutically acceptable salt thereof, wherein upon administration the composition slows the progression of psoriatic arthritis and/or symptoms thereof of the subject.

8. The method of any one of claims 1-7, wherein the SMAD7 antisense oligonucleotide or a pharmaceutically acceptable salt thereof comprises one or more O,O-linked phosphorothioate linkages.

9. The method of any one of claims 1-7, wherein all internucleoside linkages of the SMAD7 antisense oligonucleotide or pharmaceutically acceptable salt thereof are O,O-linked phosphorothioate linkages.

10. The method of any one of the above claims, wherein the pharmaceutical composition is a topical dosage form.

11. The method of claim 10, wherein the topical dosage form is formulated as a gel, a cream, an ointment, a patch, or a liquid dosage form.

12. The method of any one of claims 1-9, wherein the pharmaceutical composition is an oral pharmaceutical composition.

13. The method of claim 12, wherein the oral pharmaceutical composition is a tablet or a capsule.

14. The method of claim 13, wherein the tablet is formulated as a minitablet or a microtablet.

15. The method of claim 13, wherein the tablet comprises minitablets, microtablets, or granulates.

16. The method of claim 13, wherein the capsule comprises minitablets, microtablets, or granulates.

17. The method of any one of claims 12-16, wherein the oral pharmaceutical composition is enteric-coated.

18. The method of any one of claims 12-16, wherein the oral pharmaceutical composition is not enteric-coated.

19. The method of any one of claims 1-9, wherein the pharmaceutical composition is a parenteral pharmaceutical composition.

20. The method of any one of claims 1-9, wherein the pharmaceutical composition is a pharmaceutical composition suitable for subcutaneous administration.

21. The method of any one of the above claims, the pharmaceutical composition further comprising a pharmaceutically acceptable carrier, adjuvant and/or excipient.

22. The method of any one of the above claims, wherein the pharmaceutical composition comprises about 10% (w/w) to about 95% (w/w) of the SMAD7 antisense oligonucleotide.

23. The method of any one of the above claims, wherein the SMAD7 antisense oligonucleotide comprises a sequence 90% to 100% identical to the sequence of 5′-GTXGCCCCTTCTCCCXGCAGC-3′ (SEQ ID NO: 1) or a pharmaceutically acceptable salt thereof, wherein X is 5-methyl 2′-deoxycytidine and complements thereof.

24. The method of claim 23, wherein the SMAD7 antisense oligonucleotide comprises the sequence of 5′-GTXGCCCCTTCTCCCXGCAGC-3′ (SEQ ID NO: 1) or a pharmaceutically acceptable salt thereof.

25. The method of claim 23, wherein the SMAD7 antisense oligonucleotide comprises the sequence of 5′-GTXGCCCCTTCTCTCXGCAGC-3′ (SEQ ID NO: 2) or a pharmaceutically acceptable salt thereof.

26. A topical formulation of a Mothers against decapentaplegic homolog 7 (SMAD7) antisense oligonucleotide comprising about 10% (w/w) to about 95% (w/w) of the oligonucleotide or a pharmaceutically acceptable salt thereof, and a pharmaceutically acceptable carrier.

27. The topical formulation of claim 26 formulated as a gel, a cream, an ointment, a liquid, or a patch dosage form.

28. The topical formulation of claim 26, wherein upon applying to a skin of a subject the formulation forms a patch.

29. The topical formulation of claim 26, wherein the formulation is suitable for treating, preventing, and/or ameliorating a skin inflammation.

30. The topical formulation of claim 29, wherein the skin inflammation is psoriatic-like lesions or psoriasis.

31. The topical formulation of claim 29 or 30, wherein the skin inflammation is a pediatric skin inflammation.

32. The topical formulation of claim 26, wherein one or more of the internucleoside linkages of the SMAD7 antisense oligonucleotide are O,O-linked phosphorothioate linkages.

33. The topical formulation of claim 26, wherein all internucleoside linkages of the SMAD7 antisense oligonucleotide are O,O-linked phosphorothioate linkages.

34. The topical formulation of any one of claims 26-33, wherein the SMAD7 antisense oligonucleotide comprises a sequence 90% to 100% identical to the sequence of 5′-GTXGCCCCTTCTCCCXGCAGC-3′ (SEQ ID NO: 1) or a pharmaceutically acceptable salt thereof, wherein X is 5-methyl 2′-deoxycytidine and complements thereof.

35. The topical formulation of claim 34, wherein the SMAD7 antisense oligonucleotide comprises the sequence of 5′-GTXGCCCCTTCTCCCXGCAGC-3′ (SEQ ID NO: 1) or a pharmaceutically acceptable salt thereof.

36. The topical formulation of claim 34, wherein the SMAD7 antisense oligonucleotide comprises the sequence of 5′-GTXGCCCCTTCTCTCXGCAGC-3′ (SEQ ID NO: 2) or a pharmaceutically acceptable salt thereof.

37. A formulation comprising a Mothers against decapentaplegic homolog 7 (SMAD7) antisense oligonucleotide or a pharmaceutically acceptable salt thereof for use in treating, preventing, and/or ameliorating a skin inflammation.

38. The formulation for use of claim 37, wherein the skin inflammation comprises psoriasis-like lesions, psoriatic lesions, or psoriasis.

39. The formulation for use of claim 37, wherein the skin inflammation is a pediatric skin inflammation.

40. The formulation for use of any one of claims 37-39 formulated as a gel, a cream, an ointment, a liquid, or a patch dosage form.

41. The formulation for use of any one of claims 37-39, wherein upon applying to a skin of a subject the formulation forms a patch.

42. The formulation for use of any one of claims 37-39, wherein the formulation is an oral pharmaceutical composition.

43. The formulation for use of claim 42, wherein the oral pharmaceutical composition is a tablet or capsule.

44. The formulation for use of claim 43, wherein the tablet is formulated as a minitablet or a microtablet.

45. The formulation for use of claim 43, wherein the tablet comprises minitablets, microtablets, or granulates.

46. The formulation for use of claim 43, wherein the capsule comprises minitablets, microtablets, or granulates.

47. The formulation for use of any one of claims 42-46, wherein the oral pharmaceutical composition is enteric-coated.

48. The formulation for use of any one of claims 42-46, wherein the oral pharmaceutical composition is not enteric-coated.

49. The formulation for use of any one of claims 37-39 formulated as a parenteral pharmaceutical composition.

50. The formulation for use of any one of claims 37-39, formulated as a pharmaceutical composition suitable for subcutaneous administration.

51. The formulation for use of claim 37, wherein one or more of the internucleoside linkages of the SMAD7 antisense oligonucleotide are O,O-linked phosphorothioate linkages.

52. The formulation for use of claim 37, wherein all internucleoside linkages of the SMAD7 antisense oligonucleotide are O,O-linked phosphorothioate linkages.

53. The formulation for use of any one of claims 37-52, wherein the SMAD7 antisense oligonucleotide comprises a sequence 90% to 100% identical to the sequence of 5′-GTXGCCCCTTCTCCCXGCAGC-3′ (SEQ ID NO: 1) or a pharmaceutically acceptable salt thereof, wherein X is 5-methyl 2′-deoxycytidine and complements thereof.

54. The formulation for use of claim 53, wherein the SMAD7 antisense oligonucleotide comprises the sequence of 5′-GTXGCCCCTTCTCCCXGCAGC-3′ (SEQ ID NO: 1) or a pharmaceutically acceptable salt thereof.

55. The formulation for use of claim 53, wherein the SMAD7 antisense oligonucleotide comprises the sequence of 5′-GTXGCCCCTTCTCTCXGCAGC-3′ (SEQ ID NO: 2) or a pharmaceutically acceptable salt thereof.

56. The formulation or method of any one of the above claims, wherein the composition or formulation is administered or is suitable for administration before and/or after symptoms of moderate to severe skin inflammation, psoriasis-like lesions, and/or psoriasis is developed.

57. The formulation or method of any one of the above claims, wherein the composition or formulation is administered or is suitable for administration about every 6 hours, about every 12 hours, about every 24 hours, about every 48 hours, about every 72 hours, every day, two-times a week, once in 2 weeks, or once a month.

58. The formulation or method of any one of the above claims, wherein the subject is refractory to a first therapy.

59. The formulation or method of claim 58, wherein the first therapy is cyclosporine, corticosteroid, and/or fumaric acid esters, or derivatives thereof.

60. The formulation or method of claim 58 or 59, wherein the subject is treated concurrently or subsequent to the first therapy.


SEQUENCE LISTING (6 sequences)

SEQ ID NO: 1 - DNA, 21 bp, Artificial Sequence
  source: /note=“Description of Artificial Sequence Synthetic oligonucleotide”
  modified_base (3)..(3): 5-methyl-2′-deoxycytidine
  modified_base (16)..(16): 5-methyl-2′-deoxycytidine
gtcgccccttctccccgcagc

SEQ ID NO: 2 - DNA, 21 bp, Artificial Sequence
  source: /note=“Description of Artificial Sequence Synthetic oligonucleotide”
  modified_base (3)..(3): 5-methyl-2′-deoxycytidine
  modified_base (16)..(16): 5-methyl-2′-deoxycytidine
gtcgccccttctctccgcagc

SEQ ID NO: 3 - DNA, 20 bp, Artificial Sequence
  source: /note=“Description of Artificial Sequence Synthetic oligonucleotide”
  modified_base (3)..(3): Nitrogenous base cytosine, 5-methylcytosine or 2′-O-methylcytosine
  modified_base (4)..(4): Nitrogenous base guanine, 5-methylguanine or 2′-O-methylguanine
  modified_base (16)..(16): Nitrogenous base cytosine, 5-methylcytosine or 2′-O-methylcytosine
  modified_base (17)..(17): Nitrogenous base guanine, 5-methylguanine or 2′-O-methylguanine
  source: /note=“See specification as filed for detailed description of substitutions and preferred embodiments”
gtcgccccttctccccgcag

SEQ ID NO: 4 - DNA, 20 bp, Artificial Sequence
  source: /note=“Description of Artificial Sequence Synthetic oligonucleotide”
  modified_base (3)..(3): 5-methyl-2′-deoxycytidine
  modified_base (16)..(16): 5-methyl-2′-deoxycytidine
gtcgccccttctccccgcag

SEQ ID NO: 5 - DNA, 21 bp, Artificial Sequence
  source: /note=“Description of Artificial Sequence Synthetic oligonucleotide”
  modified_base (3)..(3): 5-methyl-2′-deoxycytidine
  modified_base (16)..(16): 5-methyl-2′-deoxycytidine
  modified_base (17)..(17): Nitrogenous base guanine, 5-methylguanine or 2′-O-methylguanine
  source: /note=“See specification as filed for detailed description of substitutions and preferred embodiments”
gtcgccccttctccccgcagc

SEQ ID NO: 6 - DNA, 21 bp, Artificial Sequence
  source: /note=“Description of Artificial Sequence Synthetic oligonucleotide”
  modified_base (3)..(3): 5-methyl-2′-deoxycytidine
  modified_base (16)..(16): 5-methyl-2′-deoxycytidine
  modified_base (17)..(17): Nitrogenous base guanine, 5-methylguanine or 2′-O-methylguanine
  source: /note=“See specification as filed for detailed description of substitutions and preferred embodiments”
gtcgccccttctctccgcagc